SEQUENCE LISTING (42 sequences)

SEQ ID NO: 1 - DNA, 23 bp, Homo sapiens
  misc_feature: PCR primer (forward)
tttgagattggccctggccccat

SEQ ID NO: 2 - DNA, 21 bp, Homo sapiens
  misc_feature: PCR primer (reverse)
gtcrttctcatctggccctaa

SEQ ID NO: 3 - DNA, 23 bp, Homo sapiens
  misc_feature: PCR primer (forward)
tttgagattggncchggcccsat

SEQ ID NO: 4 - DNA, 2471 bp, Homo sapiens
  misc_feature: cDNA
tgtccctaaaatcatctcagtacttggcacactgacttaagatgtggggtgggggagcatcccttaacacattctttgttttcctggtaaatactggtggaacaagacagctgagaatgtatgacatctgaccatgaacatatgacagctgtttgtgccagtcatgtccaaacccatggctctcaactccagatccaaaaactctccccatgttttagacctcccacaccagcatttaggatttcttcctctataatcttgctgggtgctggtcttgcagggccatctactggggataggtggtttggggtctcagtggtgggcaccggcttgttcttgcctcctctgcagctcctcttgccgcctcgcctgctgttcactcatgcaatccttgaacgcctgcacctgtggctggcattgccgccagtcctggtgctgggccatgcactcctgcactgcaaagtgggaggcagcacagccagagcgggagatcagctggtccagcgggtcctcctcctcatcgtctttcttcaccgttgggtccaggtatggccttgagggactgaggttgacatcctggggatggggagtcgaacaggtgggagaagagggccggaactccggccgggattccggatcagcattctttctgcctatatttcaaattacgcatttgccaatgttttccatggctggaagtacatgtttggtcttgtgattcccttgggagttttgcaagcaattgcaatgtattttcttcctccaagccctcggtttctggtgatgaaaggacaagagggagctgctagcaaggttcttggaaggttaagagcactctcagatacaactgaggaactcactgtgatcaaatcctccctgaaagatgaatatcagtacagtttttgggatctgtttcgttcaaaagacaacatgcggacccgaataatgataggactaacactagtattttttgtacaaatcactggccaaccaaacatattgttctatgcatcaactgttttgaagtcagttggatttcaaagcaatgaggcagctagcctcgcctccactggggttggagtcgtcaaggtcattagcaccatacctgccactcttcttgtagaccatgtcggcagcaaaacattcctctgcattggcttgctaaatgctggattaagccacactgaataccagatagtcacagaccctggggacgtcccagcttttttgaaatggctgtccttagccagcttgcttgtttatgttgctgctttttcaattggtctaggaccaatgccctggctggtgctcagcgagatctttcctggtgggatcagaggacgagccatggctttaacttctagcatgaactggggcatcaatctcctcatctcgctgacatttttgactgtaaatcttattggcctgccatgggtgtgctttatatatacaatcatgagtctagcatccctgctttttgttgttatgtttatacctgagacaaagggatgctctttggaacaaatatcaatggagctagcaaaagtgaactatgtgaaaaacaacatttgttttatgagtcatcaccaagaagaattagtgccaaaacagcctcaaaaaagaaaaccccaggagcagctcttggagtgtaacaagctgtgtggtaggggccaatccaggcagctttctccagagacctaatggcctcaacaccttctgaacgttggatagtgccagaacacttaggagggtgnacctaatggcctcaacaccttctgaacgtggatagtgccagaacacttaggagggtgtctttggaccaatgcatagttgcgactcctgtgctctcttttcagtgtccatggaactggttttgaaaaaacactcctgaaattgataaaancagcctttaacccccctccctcccccagaaaggaacccccncaaaggtttanntgaangtnacaaaggtccccctaagtttgattctccctttttttccctngaagccaaggaatttatcccnnggtttttttaaaaaaaaaaaaaaaaaagttttanccntgggcttggggttttttaaataacttttccctaacccttccccttccccacnnaganatcccannccccccccnttggaaaatanaaacctaatgntttccccccctnaaatttggaaaaaaanaattcnaaccccttccccnggnnncccctttaaagcccctaatggttttaatggtttttaatnggggaangggcccaantttccccccccaagcctttttttattttaatggccnaanaaccnccccccaagtttngggtccccctggggaaacaatgaanggggtttttncnnttttccccttgggccccttaccccaaaaaaanacttaacctttggcaccngggnaaatttcttttaaaccccccaaaaaaaaaaacnccntaaaacccccccttttttc

SEQ ID NO: 5 - protein, 526 aa, Homo sapiens
  misc_feature: Deduced amino acid sequence of GLUT8
MTSDHEHMTAVCASHVQTHGSQLQIQKLSPCFRPPTPAFRISSSIILLGAGLAGPSTGDRWFGVSVVGTGLFLPPLQLLLPPRLLFTHAILERLHLWLALPPVLVLGHALLHCKVGGSTARAGDQLVQRVLLLIVFLHRWVQVWPZGTEVDILGMGSRTGGRRGPELRPGFRISILSAYISNYAFANVFHGWKYMFGLVIPLGVLQAIAMYFLPPSPRFLVMKGQEGAASKVLGRLRALSDTTEELTVIKSSLKDEYQYSFWDLFRSKDNMRTRIMIGLTLVFFVQITGQPNILFYASTVLKSVGFQSNEAASLASTGVGVVKVISTIPATLLVDHVGSKTFLCIGLLNAGLSHTEYQIVTDPGDVPAFLKWLSLASLLVYVAAFSIGLGPMPWLVLSEIFPGGIRGRAMALTSSMNWGINLLISLTFLTVNLIGLPWVCFIYTIMSLASLLFVVMFIPETKGCSLEQISMELAKVNYVKNNICFMSHHQEELVPKQPQKRKPQEQLLECNKLCGRGQSRQLSPET

SEQ ID NO: 6 - DNA, 19 bp, Homo sapiens
  misc_feature: PCR primer (forward)
tccatggctggaagtacat

SEQ ID NO: 7 - DNA, 19 bp, Homo sapiens
  misc_feature: PCR primer (reverse)
taagtgttctggcactatc

SEQ ID NO: 8 - DNA, 18 bp, Homo sapiens
  misc_feature: PCR primer (forward)
tcaacatccacatgaact

SEQ ID NO: 9 - DNA, 21 bp, Homo sapiens
  misc_feature: PCR primer (reverse)
tgaaaaagcagcaacataaac

SEQ ID NO: 10 - DNA, 18 bp, Homo sapiens
  misc_feature: PCR primer (forward)
tgggctccaagcagatgc

SEQ ID NO: 11 - DNA, 18 bp, Homo sapiens
  misc_feature: PCR primer (reverse)
ggcttcgctggctcccac

SEQ ID NO: 12 - protein, 16 aa, Homo sapiens
  misc_feature: C-terminus of GLUT used to generate polyclonal antibody
NKLCGRGQSRQLSPET

SEQ ID NO: 13 - protein, 82 aa, Homo sapiens
  misc_feature: GLUT1
FEVGPGPIPWFIVAELFSQGPRPAAIAVAGFSNWTSNFIVGMCFQYVEQLCGPYVFIIFTVLLVLFFIFTYFKVPETKGRTF

SEQ ID NO: 14 - protein, 82 aa, Homo sapiens
  misc_feature: GLUT2
FEIGPGPIPWFMVAEFFSQGPRPAALAIAAFSNWTCNFIVALCFQYIADFCGPYVFFLFAGVLLAFTLFTFFKVPETKGKSF

SEQ ID NO: 15 - protein, 82 aa, Homo sapiens
  misc_feature: GLUT3
FEIGPGPIPWFIVAELFSQGPRPAAMAVAGCSNWTSNFLVGLLFPSAAHYLGAYVFIIFTGFLITFLAFTFFKVPETRGRTF

SEQ ID NO: 16 - protein, 82 aa, Homo sapiens
  misc_feature: GLUT4
FEIGPGPIPWFIVAELFSQGPRPAAMAVAGFSNWTSNFIIGMGFQYVAEAMGPYVFLLFAVLLLGFFIFTFLRVPETRGRTF

SEQ ID NO: 17 - protein, 82 aa, Homo sapiens
  misc_feature: GLUT8
FSIGLGPMPWLVLSEIFPGGIRGRAMALTSSMNWGINLLISLTFLTVDLIGLPWVCFIYTIMSLASLLFVVMFIPEDKGDAF

SEQ ID NO: 18 - protein, 82 aa, Homo sapiens
  misc_feature: GLUT5
HALGPSPIPALLITEIFLQSSRPSAFMVGGSVHWLSNFTVGLIFPFIQEGLGPYSFIVFAVICLLTTIYIFLIVPETKAKTF

SEQ ID NO: 19 - protein, 82 aa, Homo sapiens
  misc_feature: GLUT7
FEIGPIPIPFFGVREWFTQIWRPGAIVCVATLDWVPNFKKGICFQSLRDFKGPYHFWAFHGVVIVWYGNYWFKVPETKGKSF

SEQ ID NO: 20 - protein, 151 aa, Homo sapiens
  misc_feature: GLUT8
WKYMFGLVIPLGVLQAIAMYFLPPSPRFLVMKGQEGAASKVLGRLRALSDTTEELTVIKIKSSLKDEYQYSFWDLFRSKDNMRTRIMIGLTLVFFVQITGQPNILFYASTVLKSVGFQSMEAASLASTGVGVVKVISTIPATLLVDREGRR

SEQ ID NO: 21 - protein, 149 aa, Homo sapiens
  misc_feature: GLUT1
WPLLLSIIFIPALLQCIVLPFCPESPRFLLINRNEENRAKSVLKKLRGTADVTHDLQEMKEESRQMMREKKVTILELFRSPAYRQPILIAVVLQLSQQLSGINAVFYYSTSIFEKAGVQQPVYATIGSGIVNTAFTVVSLFVVERAGRR

SEQ ID NO: 22 - protein, 149 aa, Homo sapiens
  misc_feature: GLUT2
WHILLGLSGVRAILQSLLLFFCPESPRYLYIKLDEEVKAKQSLKRLRGYDDVTKDINEMRKEREEASSEQKVSIIQLFTNSSYRPQILVALMLHVAQQFSGINGIFYYSTSIFQTAGISKPVYATIGVGAVNMVFTAVSVFLVEKAGRR

SEQ ID NO: 23 - protein, 147 aa, Homo sapiens
  misc_feature: GLUT3
WPLLLGFTILPAILQSAALPFCPESPRFLLINRKEENAKQILQRLWGTQDVVSQDIQEMKDESARMSQEKQVTVLELFRVSSYRQPIIISIVLQLSQQGINAVFYYSTGIFKDAGVQEPIYATIGAGVVNTIFTVVSLFLVERAGRR

SEQ ID NO: 24 - protein, 149 aa, Homo sapiens
  misc_feature: GLUT4
WPLLLGLTVLPALLQLVLLPFCPESPRYLYIIQNLEGPARKSLKRLTGWADVSGVLAELKDEKRKDERERPLSLLQLLGSRTHRQPLIIAVVLQLSQQLSGINAVFYYSTSIFETAGVGQPAYATIGAGVVNTVFTLVSVLLVERAGRR

SEQ ID NO: 25 - protein, 151 aa, Homo sapiens
  misc_feature: GLUT5
WPILLGLTGVPAALQLLLLPFFPESPRYLLIQKKDEAAAKKALQTLRGWDSVDREVAEIRQEDEAEKAAGFISVLKLFRMRSLRWQLLSIIVLMGGQQLSGVNAIYYYADQIYLSAGVPEEHVQYVTAGTGAVNVVMTFCAVFVVELLGRR

SEQ ID NO: 26 - protein, 26 aa, Escherichia coli
RRAVFLGMLLQAMQQFTGMNIIMYYA

SEQ ID NO: 27 - protein, 26 aa, Saccharomyces sp.
TLRMFTGIALQAFQQFSGINFIFYYG

SEQ ID NO: 28 - protein, 26 aa, Nicotiana tabacum
RPHLTMAIMIPFFQQLTGINVIMFYA

SEQ ID NO: 29 - protein, 18 aa, Homo sapiens
  misc_feature: Synthetic peptide
EELVPKQPQKRPQELLEC

SEQ ID NO: 30 - DNA, 2649 bp, Homo sapiens
  misc_feature: GLUT8 nucleotide sequence (with C at 1129)
acattagctttgcccgaagtttttccccacactcttctttagcatgctattatggggaaagtgaccactcctgggagcgggggtggtcggggcggtttggtggcggggaagcggctgtaacttctacgtgaccatggtacctgttgaaaacaccgagggccccagtctgctgaaccagaaggggacagccgtggagacggagggcagcggcagccggcatcctccctgggcgagaggctgcggcatgtttaccttcctgtcatctgtcactgctgctgtcagtggcctcctggtgggttatgaacttgggatcatctctggggctcttcttcagatcaaaaccttattagccctgagctgccatgagcaggaaatggttgtgagctccctcgtcattggagccctccttgcctcactcaccggaggggtcctgatagacagatatggaagaaggacagcaatcatcttgtcatcctgcctgcttggactcggaagcttagtcttgatcctcagtttatcctacacggttcttatagtgggacgcattgccataggggtctccatctccctctcttccattgccacttgtgtttacatcgcagagattgctcctcaacacagaagaggccttcttgtgtcactgaatgagctgatgattgtcatcggcattctttctgcctatatttcaaattacgcatttgccaatgttttccatggctggaagtacatgtttggtcttgtgattcccttgggagttttgcaagcaattgcaatgtattttcttcctccaagccctcggtttctggtgatgaaaggacaagagggagctgctagcaaggttcttggaaggttaagagcactctcagatacaactgaggaactcactgtgatcaaatcctccctgaaagatgaatatcagtacagtttttgggatctgtttcgttcaaaagacaacatgcggacccgaataatgataggactaacactagtattttttgtacaaatcactggccaaccaaacatattgttctatgcatcaactgttttgaagtcagttggatttcaaagcaatgaggcagctagcctcgcctccactggggttggagtcgtcaaggtcattagcaccatccctgccactcttcttgtagaccatgtcggcagcaaaacattcctctgcattggctcctctgtgatggcagcttcgttggtgaccatgggcatcgtaaatctcaacatccacatgaacttcacccatatctgcagaagccacaattctatcaaccagtccttggatgagtctgtgatttatggaccaggaaacctgtcaaccaacaacaatactctcagagaccacttcaaagggatttcttcccatagcagaagctcactcatgcccctgagaaatgatgtggataagagaggggagacgacctcagcatccttgctaaatgctggattaagccacactgaataccagatagtcacagaccctggggacgtcccagcttttttgaaatggctgtccttagccagcttgcttgtttatgttgctgctttttcaattggtctaggaccaatgccctggctggtgctcagcgagatctttcctggtgggatcagaggacgagccatggctttaacttctagcatgaactggggcatcaatctcctcatctcgctgacatttttgactgtaactgatcttattggcctgccatgggtgtgctttatatatacaatcatgagtctagcatccctgctttttgttgttatgtttatacctgagacaaagggatgctctttggaacaaatatcaatggagctagcaaaagtgaactatgtgaaaaacaacatttgttttatgagtcatcaccaagaagaattagtgccaaaacagcctcaaaaaagaaaaccccaggagcagctcttggagtgtaacaagctgtgtggtaggggccaatccaggcagctttctccagagacctaatggcctcaacaccttctgaacgtggatagtgccagaacacttaggagggtgtctttggaccaatgcatagttgcgactcctgtgctctcttttcagtgtcatggaactggttttgaagagacactctgaaatgataaagacagcctttaatccccctcctccccagaaggaacctcaaaaggtagatgaggtacaaggtcctaagtgatctctttttctgagcaggatatcaggttaaaaaaaaaaagttactggctggtttaatactttctaccttcttcacagagcagcctttgaatagactatgtcctagtgaagacatcaacctccgccttaagctatgtatgtatggaggccagtcgcagctttattatgcagacacacaagtggtctggacatgagggtacagtttctgcctaccaagacactacttgcactggatcttacgcaaaaaagaaccagaacacacagtgtggacaactgcccatatattctatctagattaggagagggtcctggctaggattttagtggtaattcctagttacattcaacaagtataaagattatagagcttattttatgaactataaactataatttaatgcaaaatatccttttatgaatttcatgttaatattgtgaaatattaaaataattcc

SEQ ID NO: 31 - DNA, 123 bp, Homo sapiens
  misc_feature: Amino terminus (nucleotides 134 to 256) of SEQ ID NO4
atggtacctgttgaaaacaccgagggccccagtctgctgaaccagaaggggacagccgtggagacggagggcagcggcagccggcatcctccctgggcgagaggctgcggcatgtttaccttc

SEQ ID NO: 32 - protein, 41 aa, Homo sapiens
  misc_feature: Amino terminus of SEQ ID NO4
MVPVENTEGPSLLNQKGTAVETEGSGSRHPPWARGCGMFTF

SEQ ID NO: 33 - DNA, 69 bp, Homo sapiens
  misc_feature: Transmembrane domain (TM)7
cgaataatgataggactaacactagtattttttgtacaaatcactggccaaccaaacatattgttctat

SEQ ID NO: 34 - protein, 23 aa, Homo sapiens
  misc_feature: Transmembrane domain (TM)7
RIMIGLTLVFFVQITGQPNILFY

SEQ ID NO: 35 - DNA, 285 bp, Homo sapiens
  misc_feature: Loop between TM9 and TM10 (nucleotides 1238 to 1514)
cacatgaacttcacccatatctgcagaagccacaattctatcaaccagtccttggatgagtctgtgatttatggaccaggaaacctgtcaaccaacaacaatactctcagagaccacttcaaagggatttcttcccatagcagaagctcactcatgcccctgagaaatgatgtggataagagaggggagacgacctcagcatccttgctaaatgctggattaagccacactgaataccagatagtcacagaccctggggacgtcccagcttttttgaaatggctg

SEQ ID NO: 36 - protein, 95 aa, Homo sapiens
  misc_feature: Loop between TM9 and TM10 (amino acids 369 to 463)
HMNFTHICRSHNSINQSLDESVIYGPGNLSTNNNTLRDHFKGISSHSRSSLMPLRNDVDKRGETTSASLLNAGLSHTEYQIVTDPGDVPAFLKWL

SEQ ID NO: 37 - DNA, 135 bp, Homo sapiens
  misc_feature: Cytochalasin B binding site (nucleotides 1556 to 1690)
ttttcaattggtctaggaccaatgccctggctggtgctcagcgagatctttcctggtgggatcagaggacgagccatggctttaacttctagcatgaactggggcatcaatctcctcatctcgctgacatttttg

SEQ ID NO: 38 - protein, 45 aa, Homo sapiens
  misc_feature: Cytochalasin B binding site (amino acids 475 to 519)
FSIGLGPMPWLVLSEIFPGGIRGRAMALTSSMNWGINLLISLTFL

SEQ ID NO: 39 - DNA, 210 bp, Homo sapiens
  misc_feature: Carboxyl terminus (nucleotides 1775 to 1984)
tttatacctgagacaaagggatgctctttggaacaaatatcaatggagctagcaaaagtgaactatgtgaaaaacaacatttgttttatgagtcatcaccaagaagaattagtgccaaaacagcctcaaaaaagaaaaccccaggagcagctcttggagtgtaacaagctgtgtggtaggggccaatccaggcagctttctccagagacc

SEQ ID NO: 40 - protein, 70 aa, Homo sapiens
  misc_feature: Carboxyl terminus (amino acids 548 to 617)
FIPETKGCSLEQISMELAKVNYVKNNICFMSHHQEELVPKQPQKRKPQEQLLECNKLCGRGQSRQLSPET

SEQ ID NO: 41 - DNA, 18 bp, Homo sapiens
  misc_feature: PCR primer (forward)
cgaagtttttccccacac

SEQ ID NO: 42 - DNA, 20 bp, Homo sapiens
  misc_feature: PCR primer (reverse)
tgtaactaggaattaccact

(19) United States
(12) Patent Application Publication
(10) Pub. No.: US 2003/0228592 A1
(43) Pub. Date: Dec. 11, 2003

(54) Human facilitative glucose transport protein GLUT8

(75) Inventors: Suzanne Dawn Rogers, Coburg (AU); James Donovan Best, Hawthorn (AU)

Correspondence Address: FOLEY AND LARDNER, SUITE 500, 3000 K STREET NW, WASHINGTON, DC, 20007, US

(73) Assignee: St. Vincent's Institute of Medical Research

(21) Appl. No.: 10/367,980

(22) Filed: Feb. 19, 2003

Related U.S. Application Data
(63) Continuation-in-part of application No. 09/509,731, filed on Jun. 9, 2000, now abandoned.
Application No. 09/509,731 is a 371 of international application No. PCT/AU98/00819, filed on Sep. 30, 1998.

(30) Foreign Application Priority Data
Oct. 1, 1997 (AU) P0 9573

(51) Int. Cl. A61K 51/00; C12Q 1/68; G01N 33/574; A61K 31/70; C07H 21/04; C12P 21/02; C12N 5/06; C07K 14/705; C07K 16/30

(52) U.S. Cl. 435/6; 435/7.23; 435/320.1; 435/325; 530/350; 530/388.1; 424/1.11; 514/23; 435/69.1; 536/23.5

(57) ABSTRACT

The present invention relates to a novel glucose transporter protein isolated from a breast cancer cell line, and to the gene encoding the protein. Detection of expression of the protein is useful as a diagnostic and staging marker in cancer. Control of expression of the protein is useful in the therapy of cancer. Furthermore, up-regulation of the protein is useful to overcome insulin resistance in non-insulin dependent diabetes mellitus.

### CROSS-REFERENCE TO RELATED PATENT APPLICATIONS

This application is a continuation-in-part of U.S. patent application Ser. No. 09/509,731, filed Jun. 9, 2000, pursuant to 35 U.S.C. §371 based on PCT/AU98/00819, filed Sep. 30, 1998, claiming priority of Australian provisional application No. PO 9573, filed Oct. 1, 1997.

### FIELD OF THE INVENTION

The present invention relates to a novel glucose transporter protein (GLUT8) and the gene encoding GLUT8. More specifically, the present invention relates to the use of GLUT8 for the detection of malignant conditions, as a staging marker in cancer and as a target useful in the treatment of cancer. Additionally, the present invention relates to the upregulation of GLUT8 in the treatment of insulin resistance in non-insulin dependent diabetes mellitus.

### BACKGROUND OF THE INVENTION

Transport of blood glucose across the plasma membrane occurs via facilitative glucose transport, catalyzed by a family of facilitative-diffusion glucose transporter molecules. The mammalian glucose transporters (GLUTs) are a group of closely-related facilitative hexose transporter proteins which are expressed in a tissue-specific manner. The pattern of expression reflects both the kinetic and substrate binding characteristics of the transporters and the sugar requirements of individual tissues. In human tissues, five members of the facilitative glucose transporter family have been identified. The cDNAs for GLUTs 1, 2, 3, 4 and 5 have been cloned and sequenced, their tissue distribution determined and their kinetic properties studied. GLUT6 had been designated as a further member of this family, but was subsequently shown to be a pseudogene. In rat liver, an isoform with strong sequence similarity to GLUT2 and designated as GLUT7 has been reported. However, no evidence that this transporter is expressed in humans has been shown (reviewed by Bell et al 1993).

Expression of the GLUT4 isoform is restricted to skeletal muscle and adipose tissue. Under basal conditions most GLUT4 protein resides in intracellular compartments. When muscle cells and adipose tissue are exposed to insulin, GLUT4 protein is translocated to the plasma membrane, where it is able to transport glucose into cells. The complex mechanisms which are involved in this process of translocation to the plasma membrane in response to insulin are still being elucidated. It is known that phosphorylation of specific amino acids controls trafficking of GLUT4. Extensive studies have concluded that mutations in the GLUT4 gene are not present in patients with non-insulin dependent diabetes mellitus (NIDDM) (Kahn, 1994). However, it is thought that defects in the insulin-regulated translocation of GLUT4 may play a part in insulin resistance associated with NIDDM.

Malignant cells are rapidly dividing, and therefore have increased glucose requirements. Oxidative metabolism is generally impaired in these cells, and tumor cells are characterized by high rates of glucose uptake, lactate formation and glycolysis. The levels of expression of both GLUT1 (erythrocyte/HepG2 glucose transporter) and GLUT3 (brain/fetal glucose transporter) isoforms can be elevated in malignant cells. For example, mRNA levels of GLUT1 and GLUT3 are significantly elevated in esophageal, stomach and colon cancers and primary brain tumors. There is much evidence to indicate that upregulation of glucose transport is a fundamental part of the malignant process. As in other tumors, malignancy in breast tumors is associated with altered metabolism and increased glucose uptake. In normal mammary epithelial cells only the GLUT1 isoform is expressed, and levels of transporter are altered by hormonal influences during lactation and weaning. Some breast tumors over-express GLUT1. The presence of GLUT4, GLUT2 and GLUT5 in breast tumor cells has also been reported.

Glucose transporter expression is remains a target for diagnosis and treatment of malignant conditions because these proteins are thought to participate in meeting the high energy requirements of malignant cells.

### SUMMARY OF THE INVENTION

There remains a need in the art to discover genes and their corresponding proteins for use in diagnosing and treating malignant conditions. Furthermore, there is a need to treat conditions, such as non-insulin dependent diabetes mellitus, where facilitative glucose transporter proteins are implicated.

To address this and other needs the present invention provides a novel facilitative glucose transporter protein (GLUT8), nucleic acids encoding GLUT8 and fragments thereof for use in diagnosing and treating malignant conditions. The invention also provides methods of treating non-insulin dependent diabetes mellitus.

One embodiment of the invention is an isolated and purified nucleic acid molecule encoding a novel facilitative glucose transporter protein which we have designated GLUT8.

Another embodiment of the invention is an isolated and purified nucleic acid molecule which hybridizes under stringent conditions to the sequence set out in SEQ ID NOs: 4 or 30.

A further embodiment of the invention is a facilitative glucose transporter protein designated GLUT8.

An additional embodiment of the invention is a method for diagnosing a malignant condition, comprising the steps of detecting expression or activity of GLUT8 in a tissue or cell, correlating the expression or activity of GLUT8 with a malignant condition, and diagnosing the malignant condition.

Another embodiment of the invention is a method of selecting a method of treatment of a malignant condition, comprising the step of measuring the ability of a proposed therapeutic agent to inhibit activity or expression of GLUT8 in a tissue or cell.

A further embodiment of the invention is an antibody directed against GLUT8, or a functional fragment thereof.

An additional embodiment of the invention is a method of treating non-insulin dependent diabetes mellitus, comprising the step of upregulating expression of GLUT8 in a tissue or cell.

Another embodiment of the invention is a method of detecting a mutation in the GLUT8 gene or regulatory sequence of a patient comprising the step of analyzing the gene or regulatory sequence for a nucleic acid change compared to that set out in SEQ ID NO: 3 or 4.

Another embodiment of the invention is a method of screening putative agents for treatment of cancer, comprising the step of measuring the ability of the agents to inhibit the activity of GLUT8 in vitro or in vivo.

A further embodiment of the invention is a method of screening putative agents for treatment of diabetes and/or insulin-resistance syndrome comprising the step of measuring the ability of the agents to upregulate or enhance the activity of GLUT8 in vitro or in vivo.

Other features, objects, and advantages of the present invention are apparent in the detailed description that follows. It should be understood, however, that the detailed description, while indicating preferred embodiments of the invention, are given by way of illustration only, not limitation. Various changes and modifications within the spirit and scope of the invention will become apparent to those skilled in the art from the detailed description.

### BRIEF DESCRIPTION OF THE DRAWINGS

FIG. 1a shows the results of Western blotting using proteins extracted from the malignant breast epithelial cell lines MCF7 and T47-D, and GLUT4 C-terminal polyclonal antibody (R820; James et al., 1989).

FIG. 1b shows the results of Northern analysis of RNA extracted from MCP7 malignant breast epithelial cells probed with GLUT4 cDNA.

FIG. 2 shows the results of Southern hybridization analysis using GLUT4 cDNA to probe RT-PCR products isolated from MCF7 cells and T47-D cells. The primers used were the degenerate primer SEQ ID NO:3 and reverse primer, SEQ ID NO:2. GLUT4 cDNA was used as positive control.

FIG. 3 shows the initial sequence alignment of the deduced amino acid sequence corresponding to the 350 bp PCR fragment with human GLUT1 (SEQ ID NO: 13), GLUT2 (SEQ ID NO: 14), GLUT3 (SEQ ID NO: 15), GLUT4 (SEQ ID NO: 16), GLUT8 (SEQ ID NO: 17), GLUT5 (SEQ ID NO: 18), and rat GLUT7 (SEQ ID NO: 19).

FIG. 4 shows genomic DNA extracted from MCF7 cells and whole blood, digested with restriction enzymes Pst 1, EcoR 1, separated on agarose gels and transferred to nylon membrane by Southern transfer. Filters were probed with the GLUTS full-length cDNA probe.

FIG. 5 shows PCR amplified products obtained using the GLUT8 specific primer pair 1 on genomic DNA from MCF7 cells. Southern blot was probed with GLUT8 cDNA.

FIG. 6 shows the sequence alignment between part of the deduced amino acid sequence of the first GLUT8 cDNA clone (SEQ ID NO: 20) and the sequences of human GLUT1 (SEQ ID NO: 21), GLUT2 (SEQ ID NO: 22), GLUT3 (SEQ ID NO: 23), GLUT4 (SEQ ID NO: 24), and GLUT5 (SEQ ID NO: 25).

FIG. 7 illustrates amino acid sequence homologies between different GLUT8 regions and corresponding regions of human GLUT1 to GLUT5, and with the binding sites of other facilitative glucose transporters:

a) The putative substrate binding site in helix 7: SEQ ID NOs: 21-25 and 20, respectively, in order of appearance;

b) The cytochalasin B binding site: SEQ ID NOs: 13-16, 18 and 17, respectively, in order of appearance; and

c) Binding sites of other glucose transporters: GLUT8 (SEQ ID NO: 20), E. coli AraE arabinose transporter (ARA; SEQ ID NO: 26), yeast SNP3 glucose transporter MST1 (YHT; SEQ ID NO: 27), and monosaccharide transport protein from Nicotiana tabacum. (SEQ ID NO: 28).

FIG. 8 shows the results of RT-PCR detection of GLUT8 in human tissues and cell lines.

FIG. 9 shows the results of Southern analysis of RT-PCR using primer pair 1, indicating preferential expression of GLUT8 in malignant breast tissue compared to normal tissue.

a) Breast tumor sample, normal breast tissue, and MCF7 cells, probed with GLUT8 (primer pair 2).

b) GLUT1 to GLUT5, GLUT8, negative control, and GLUT1 cDNA (positive control), probed with GLUT1 (primer pair 1).

c) GLUT1 to GLUT5, GLUT8, negative control and GLUT4 plasmid (positive control) probed with GLUT4 (primer pair 1).

FIG. 10 shows the results of immunocytochemical detection of GLUT8 in cultured malignant breast cells and in normal and malignant breast tissue

a) Pre-immune serum.

b) Breast tumor sample incubated with immune serum.

c) Normal breast tissue incubated with non-immune serum.

FIG. 11 shows immunocytochemical detection of GLUT5 in MCF7 cells under basal conditions, and following insulin treatment for 15 min. GLUT8 antiserum is used at 1/100 and 1/300 dilutions. Specific staining is competed by competitive, but not non-competitive peptide.

FIG. 12 shows a Western Blot of a 50 kDa protein species detected by GLUT8 antisera in MCF7 and T47-D cells by affinity purified antiserum (AP) in MCF7 cells.

FIG. 13 shows MCF7 protein extracts following membrane fractionation and deglycosylation.

FIG. 14 demonstrates that the GLUT4 monoclonal antibody, 1F8 does not detect a protein of 50 kDa in MCF7 cells. Rat gastroonemius muscle protein extracts are used as a positive control for GLUT4 protein.

FIG. 15 shows GLUT8 protein being detected in both the rat adipose tissue and skeletal muscle by GLUT8 antiserum.

FIG. 16a shows the detection of GLUT8 protein in human adipose tissue and skeletal muscle.

FIG. 16b Immunohistochemical detection of GLUT8 in human skeletal muscle.

FIG. 17 shows in vitro transcription/translation of GLUT4 and GLUT8 mRNA in the presence and absence of microsomes.

FIG. 18 shows the effects of preincubation with insulin on imnmunocytochemical staining of MCF7 breast tumor cells:

a) Pre-immune serum,

b) Immune serum without insulin pretreatment,

c) Immune serum after preincubation with 10 nM insulin for 15 min.

FIG. 19 shows immunofluorescence staining of GLUT8 in MCF7 cells under basal conditions, and after long-term exposure to insulin.

FIG. 20 shows a Northern blot analysis of polyA RNA extracted from MCF7 cells. Specific transcripts of approximately 4.4 and 2.5 kb were detected with GLUT8 cDNA.

### DETAILED DESCRIPTION OF THE PREFERRED EMBODIMENTS

This invention originated from the hypothesis that the hormonal factors, such as estrogen, which influence breast cancer progression and cell proliferation might be involved in regulation of heterogeneous glucose transporter expression in breast cancer. The present invention is based on the discovery that a new type of glucose transporter protein, which is related to but distinct from GLUT4, is present in a malignant breast epithelial cell line, and that this transporter protein is also present in human skeletal muscle and adipose tissue cells.

According to a first aspect, the invention provides a nucleic acid molecule encoding a novel facilitative glucose transporter protein designated GLUT8. The nucleic acid sequence may be genomic DNA, cDNA, or RNA, and may be single stranded or double stranded. Preferably, the nucleic acid is cDNA. More preferably, the nucleic acid molecule has the sequence as set out in SEQ ID NO:4 or SEQ ID NO: 30. SEQ ID NOs: 4 and 30 are identical with the exception of nucleotide 1129. Due to an inability to resolve the nucleotide residue at position 1129, it is unclear whether this residue is A or C. This ambiguity does not affect the resulting amino acid sequence (SEQ ID NO: 5). As such, both sequences are provided. SEQ ID NO: 4 is shown with C at position 1129 and SEQ ID NO: 30 is shown an A at position 1129.

The invention also encompasses nucleic acid molecules that encode useful fragments of the full-length GLUT8 proteins. For example, because glucose transporter proteins have little or no sequence homology in their amino (SEQ ID NO: 31) and carboxy (SEQ ID NO: 39) termini, this region may be used for specific antibody generation. (See Example 8 for production of antibodies directed to the C-terminus of GLUT8.) Furthermore, intracellular targeting motifs are also believed to be present in the carboxy and amino termini region and thus these regions may be important for targeting GLUT8 to specific cellular locations, with the potential aim of substrate or drug delivery to cells expressing the protein on the cell surface.

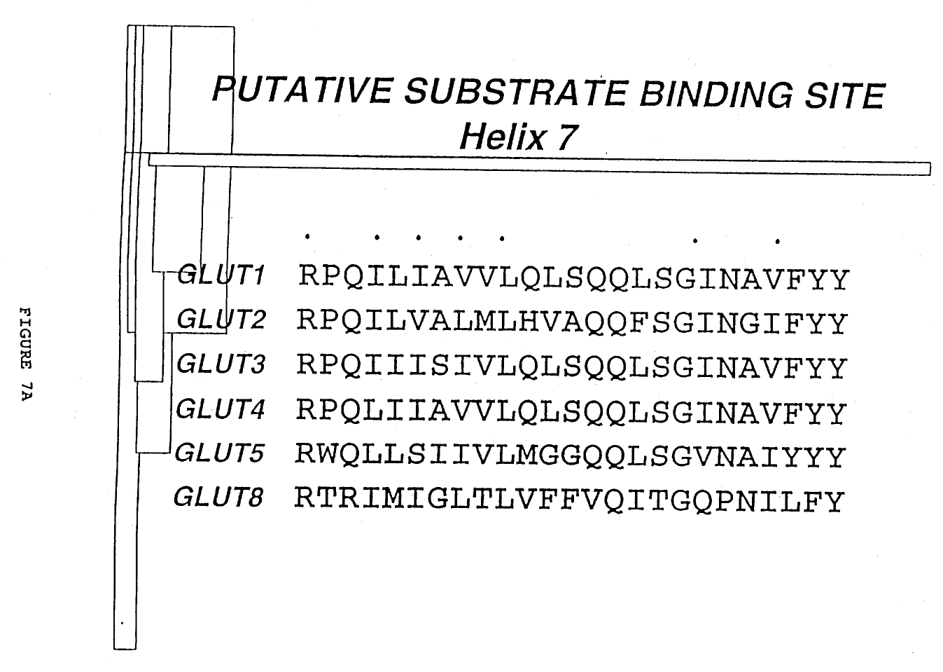

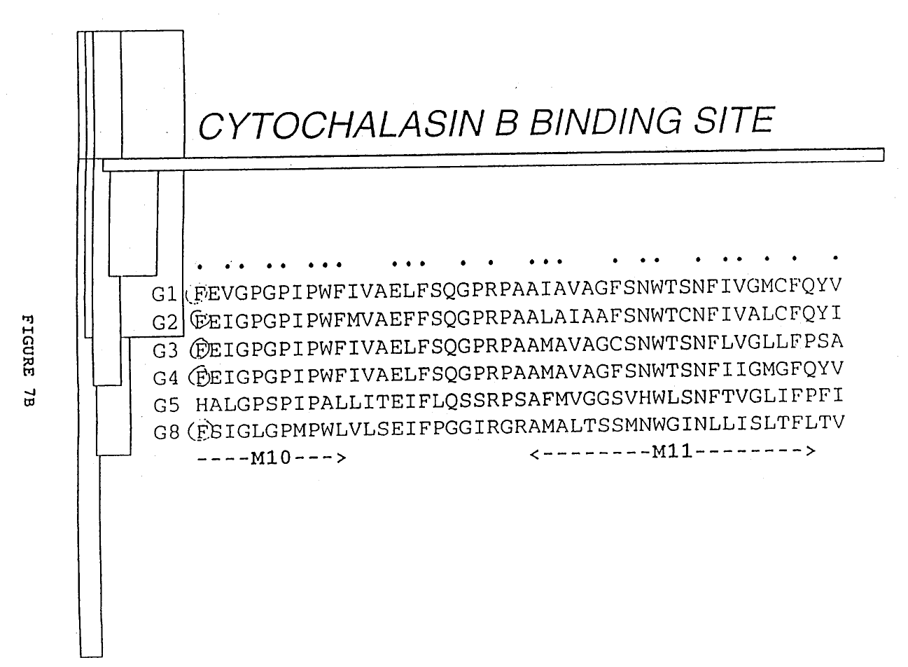

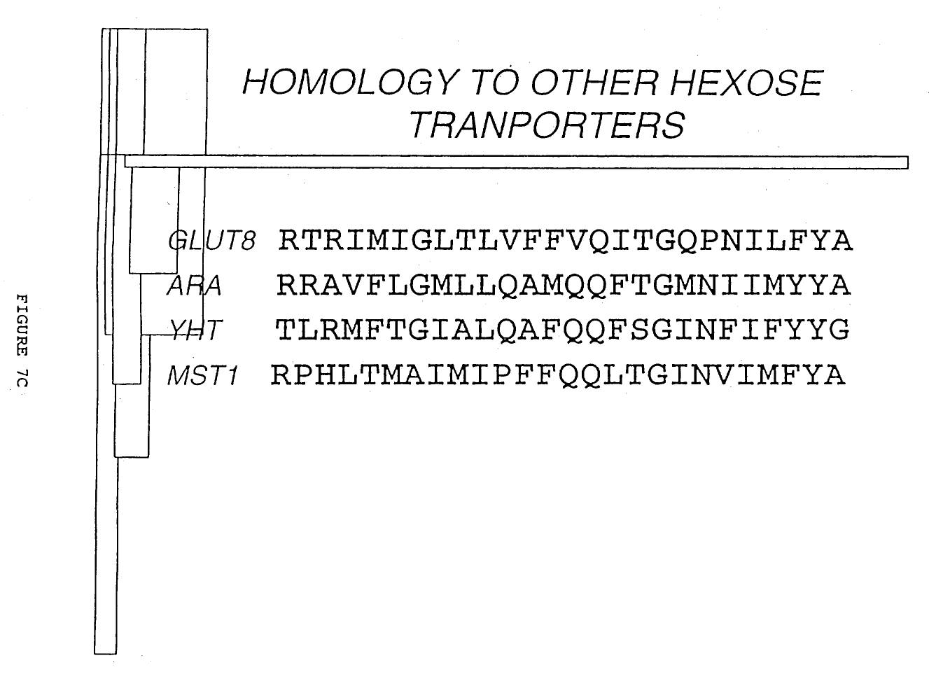

A nucleic acid encoding transmembrane domain 7 (TM7), the amino acid sequence of which is shown in FIG. 7a, is a further nucleotide fragment (SEQ ID NO: 33), is encompassed by the invention. TM7 contains the exofacial substrate binding site of GLUT proteins (Arbuckle et al., 1996). Motifs in this region determine substrate specificity and kinetic properties of transporters. These motifs may be important for binding and uptake of novel substrates, such as glucose analogues, which may in turn be used for diagnostic or therapeutic purposes.

Another nucleic acid fragment of the invention is the loop between transmembrane domain 9 (TM9) and transmembrane domain 10 (TM10) provided in SEQ ID NO: 37. This region encompasses a proposed large extracellular loop of the GLUT8 protein. This loop region contains several potential N-linked glycosylation sites. GLUT1 activity is influenced by glycosylation status, therefore, by analogy, alteration of GLUT8 glycosylation status can be used to alter the kinetics of substrate transport (Asano et al., 1991). Additionally, because this loop region is not homologous with the corresponding region of other GLUT family members, it may also be important for the specific targeting of GLUT8 in monoclonal antibody therapy.

Another useful nucleic fragment is that which encodes the cytochalasin B binding site (SEQ ID NO:38), shown in FIG. 7b. Cytochalasin B is a glucose transport inhibitor that binds at the endofacial substrate binding site of GLUT proteins, located within the region of TM 10 and TM 11 (Garcia et al., 1992). This binding site may be used for inhibition of substrate binding and uptake and to determine other potential substrates or inhibitors of GLUT8.

Although the invention is described in detail in relation to cDNA, the person skilled in the art will be able to utilize known methods in order to prepare nucleic acid sequences of other kinds.

The person skilled in the art would also appreciate that the present invention provides a nucleic acid molecule or fragment thereof which hybridizes under stringent conditions to the sequence set out in SEQ ID NO:4. “Stringent conditions” are those that (1) employ low ionic strength and high temperature for washing, for example, 0.015 M NaCl/0.0015 M sodium citrate/0.1% NaDodSO₄ (SDS) at 50° C., or (2) employ during hybridization a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Another example is use of 50% formamide, 5×SSC (0.75 M NaCl, 0.075 N sodium citrate), 50 mM sodium phosphate (pH 6.8), 0.1% sodium pyrophosphate, 5× Denhardt's solution, sonicated salmon sperm DNA (501 g/ml), 0.1% SDS, and 10% dextran sulfate at 42° C., with washes at 42° C. in 0.2×SSC and 0.1% SDS.

In a second aspect, the invention provides the transporter protein, GLUT8, and biologically-active fragments, analogues and derivatives thereof. One embodiment of the invention comprises GLUT8 fragments, such as those comprising the carboxy (SEQ ID NO: 40) and amino (SEQ ID NO: 32) termini, TM7 (SEQ ID NO: 34), the loop between TM9 and TM10 (SEQ ID NO: 36), and the cytochalasin B binding site (SEQ ID NO: 38). These protein regions are described above. In another embodiment, the transporter protein preferably has greater than 70% sequence homology with the amino acid sequence set out in SEQ ID NO:5. More preferably, greater than 80% sequence homology. Even more preferably, greater than 95%, and most preferably, the protein has the amino acid sequence as set out in SEQ ID NO:5. Using methods known in the art, the skilled person will be able to identify biologically-active fragments or analogues of the protein described in detail herein. For example, routine methodology can be used to probe the known biological activity of GLUT8 to determine level of GLUT8 in sample. For example, see Thomas et al. (1993). Methods such as site-directed mutagenesis may be used to prepare nucleic acid sequences encoding substitutions, deletions and additions to the naturally-occurring gene and amino acid sequences.

In a third aspect, the invention provides a method of diagnosis of a malignant condition, comprising the step of detecting expression or activity of GLUT8 in a tissue or cell.

In a fourth aspect, the invention provides a method of monitoring of efficacy of treatment of a malignant condition, comprising the step of detecting activity or expression of GLUT8 in a tissue or cell.

According to a fifth aspect, the invention provides a method of selecting a method of treatment of a malignant condition, comprising the step of measuring the ability of a proposed therapeutic agent to inhibit activity or expression of GLUT8 in a tissue or cell.

It is contemplated that non-utilizable glucose analogues targeted to the malignant tissue will be particularly suitable for inhibiting expression and/or activity of GLUT8 for treatment of cancer.

It is also contemplated that anti-sense nucleic acid sequences directed against the GLUT8 nucleic acid sequence will be useful for inhibiting expression of GLUT8.

It will be further contemplated that dominant/negative mutants of GLUT8 nucleic acids or protein which retains some function will be useful for inhibiting the growth of breast cancer.

Preferably the malignant condition is breast cancer, but it is also contemplated that methods of the invention will be useful for treatment of prostate cancer and other epithelial cell cancers, particularly skin cancers, including malignant melanoma, and colon cancers.

In a sixth aspect the invention provides an antibody directed against GLUT8. The antibody may be polyclonal or monoclonal, but is preferably polyclonal. Preferably, the antibody is directed against a portion of the C-terminal region of GLUT8. More preferably, the antibody is directed against one or more epitopes present in the sequence NKLCGRGGQSRQLSPET (SEQ ID NO:12).

Methods for production and screening of monoclonal antibodies are very well known in the art. The antibodies of the invention are useful for assay of GLUT8 protein, for example by radioimmunoassay, ELISA assay, and by immunocytochemical detection. Some antibodies of the invention have the ability to inhibit the activity of GLUT8, and the person skilled in the art will readily be able to identify whether or not a given antibody has such inhibitory activity. It will be clearly understood that fragments such as Fv, Fab and F(ab)₂ and analogues such as ScFv and humanised antibodies which are able to bind to and/or inhibit GLUT8 are within the scope of the invention. Again methods for production of such fragments and analogues are well known in the art. See for example, Australian Patent No 690528, International Patent Application No PCT/AU93/00491 and No PCT/AU98/00212, and references cited therein.

Because of its homology with GLUT4, and because of the known involvement of GLUT4 and the known effect of insulin on translocation of GLUT4 to the plasma membrane, it is contemplated that upregulation of GLUT8 expression will be useful to overcome insulin resistance in non-insulin dependent diabetes mellitus.

Thus in a seventh aspect the invention provides a method of treatment of non-insulin dependent diabetes mellitus, comprising the step of upregulating expression of GLUTS in a tissue or cell. Preferably the tissue is skeletal muscle and/or adipose tissue. For example tissue-localised gene therapy may be used for expression of GLUTS in skeletal muscle in order to stimulate glucose uptake.

It will be appreciated by the skilled person that mutations in the GLUT8 gene or regulatory sequences may be involved in NIDDM. As such, genetic mutation of GLUT8 may have a causal or exacerbating effect with regards to NIDDM. Thus, the person skilled in the art would appreciate that mutations in the GLUT8 gene or regulatory regions may be corrected by gene therapy.

Accordingly, an eighth aspect the invention provides a method of detecting a mutation in the GLUT8 gene or regulatory sequence of a patient comprising the step of analyzing the gene or regulatory sequence for a nucleic acid change compared to that set out in SEQ ID NO: 3 or 4. Preferably, the patient is a NIDDM patient, and the method of detection is single stranded conformational polymorphism (SSCP) or other genetic analysis procedure known in the art.

In a ninth aspect the invention provides a method of screening putative agents for treatment of cancer, comprising the step of measuring the ability of the agents to inhibit the activity of GLUT8 in vitro or in vivo.

In a tenth aspect the invention provides a method of screening putative agents for treatment of diabetes and/or insulin-resistance syndrome comprising the step of measuring the ability of the agents to upregulate or enhance the activity of GLUT8 in vitro or in vivo.

In the third, fourth and fifth aspects of the invention, expression of GLUT8 may be detected by a variety of different means, including but not limited to immunocytochemistry, hybridization analysis, PCR, RT-PCR and the like, using a sample of tissue or of biological fluid suspected to contain cancer cells.

Activity of GLUT8 in vivo may for example be detected by positron emission tomography scanning using a hexose labelled with a fluorescent marker; preferably the hexose is a glucose analogue or hexose specifically transported by GLUT8.

It will be clearly understood that for the purposes of this specification the word “comprising” is to be understood to mean “including, but not limited to”.

Unless specifically described herein, methods utilized are generally known in the art, for example, by reference to Sambrook et al (1989).

It will be apparent to the person skilled in the art that while the invention has been described in some detail for the purposes of clarity and understanding, various modifications and alterations to the embodiments and methods described herein may be made without departing from the scope of the inventive concept disclosed in this specification.

### EXAMPLE 1

### Identification of a GLUT4-Related Sequence in a Breast Cancer Cell Line

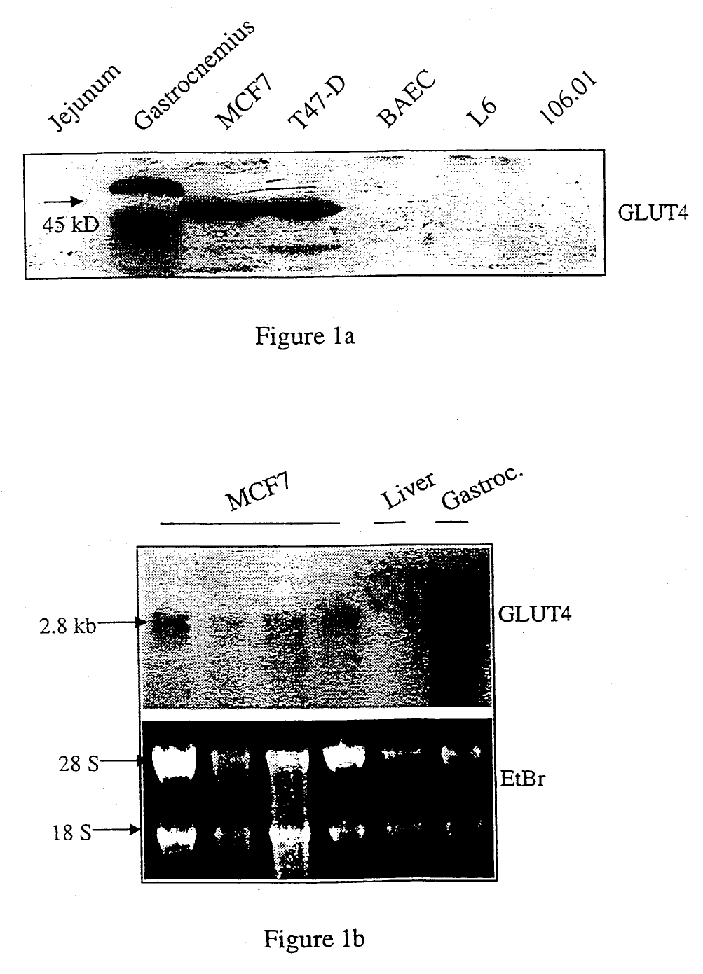

Expression of GLUT4 is normally tightly restricted to skeletal muscle and adipose tissue. Using standard methods we detected a protein of similar molecular weight (49 kd) to GLUT4 in two malignant breast epithelial cell lines, MCF7 and T47-D Soule et al. 1973; Keydar et al 1979, respectively). Briefly, total cell homogenates (1.5 μg/μl) were subjected to SDS-polyacrylamide electrophoresis. Proteins were transferred to membranes and after blocking for non-specific binding were incubated with GLUT4 polyclonal anti-sera diluted 1:300 (James et al. 1989). Immunoreactive proteins were detected using ¹²⁵I-labelled Protein A, and the results are shown in FIG. 1a. Rat jejunum and gastrocnemius muscle, bovine aortic endothelial cells (BAEC), L6 rat myoblast cells, and 106.01 rat malignant bone cells were used for comparison. A GLUT4 like protein was detected in the two malignant breast cell lines and in the skeletal muscle sample, but not in other tissues or cells.

Northern analysis showed a specific transcript of 2.8 kb when RNA extracted from MCF7 cells was probed with the GLUT4 cDNA (cDNA probe provided by D. E. James). Rat liver and gastrocnemius muscle RNA samples were used as negative and positive controls respectively. The results are summarised in FIG. 1b. This transcript was smaller than would be expected for GLUT4 (2.8 kb compared to 3.5 kb) for a human transcript.

### EXAMPLE 2

### The Protein is Distinct from GLUT 4

To confirm the presence of GLUT4 in MCF7 cells, we designed primers based on the GLUT4 sequence and performed RT-PCR reactions on RNA extracted from MCF7 and T47-D cells. MCF-7 cells were purchased from the American Type Culture Collection and maintained in RPMI 1640 medium with L-glutamine (Life Technologies), 10% fetal bovine serum (FBS) (CSL Biosciences), and 50 nM insulin (Novo Nordisk). Total RNA was extracted using Trizol reagent (Life Technologies), according to the manufacturer's instructions. Reverse transcriptase reactions were performed with AMV Reverse Transcriptase and Oligo(dT)15 primers (Promega). using conditions recommended by the manufacturer. GLUT4 specific RT-PCR primers were as follows:

| | |
|---|---|
| Forward primer: | |
| TTTGAGATTGGCCCTGGCCGCAT | SEQ ID NO: 1 |
| Reverse primer: | |
| GTC (AG)TTCTCATCTGGCCCTAA | SEQ ID NO: 2 |

PCR was performed at an annealing temperature of 49° C. and 30 sec extension for 40 cycles, using Taq DNA polymerase purchased from Boehringer. We were unable to confirm the presence of GLUT4 by this method.

### EXAMPLE 3

### Isolation and Sequencing of a PCR Fragment

On the assumption that the protein detected by Western and Northern blots might therefore be a GLUT4-like protein, we performed further PCR reactions. Using reduced annealing temperatures and degenerate primers, a PCR product was obtained. The PCR primers were designed to encompass the region from the transmembrane domain 10 (TM10) to the C-terminus of a proposed facilitative glucose transporter. All members of the GLUT family have a high degree of homology of TM10. It was expected that a GLUT4-like protein would possess some to GLUT4 in the C-terminal region, particularly as the GLUT4 polyclonal antibody which we used for the experiments in Example 1 was designed to interact with the C-terminus. Primers used in these experiments were:

Forward primer:

TTTGAGATTGGNCC (TAC) GGCCC (CG) AT SEQ D NO: 3

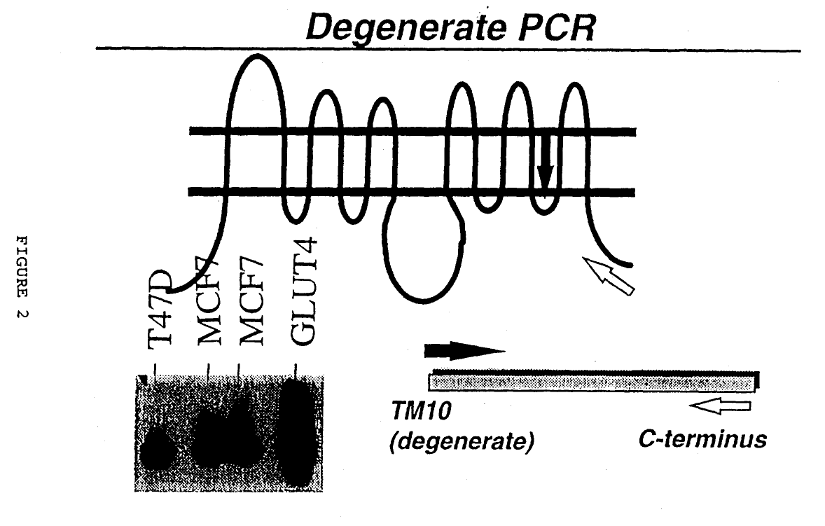

and reverse primer of SEQ ID NO:2 as defined above. The PCR reaction conditions utilised touchdown PCR, with the first 5 cycles at an annealing temperature of 37° C. followed by 35 cycles at an annealing temperature of 49° C. The extension time was 30 sec. For these experiments, the Expand High Fidelity PCR System (Boehringer) was used. A 350 bp PCR product was obtained. This PCR product hybridized to the GLUT4 cDNA by Southern analysis, as shown in FIG. 2.

The 350 bp PCR fragment was purified from agarose gels and sequenced by direct incorporation using the fmol DNA Sequencing System (Promega). cDNAs were labeled with [α³²P]dCTP and the Random Primed DNA labeling kit (Roche Molecular Biochemicals).

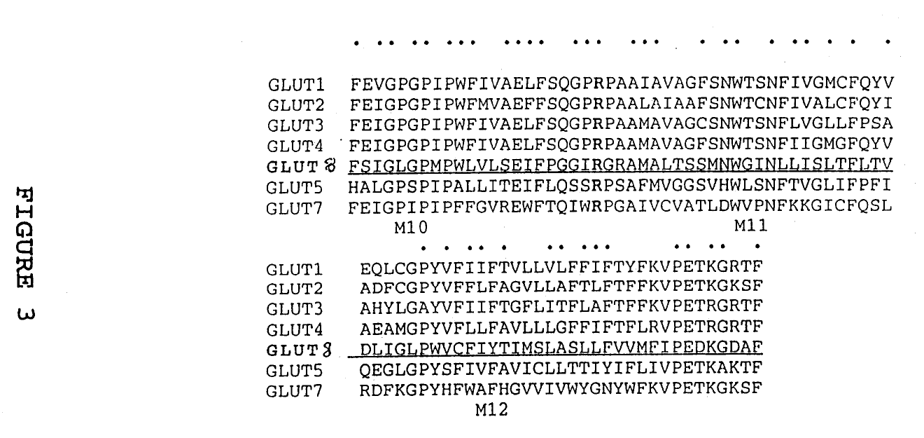

The 350 bp PCR fragment was determined to be distinct from nucleic acid encoding GLUT4. Approximately 250 bp of nucleotide sequence were obtained, and FIG. 3 shows initial sequence alignments obtained using the deduced amino acid sequence. This showed that the new protein, designated GLUT8, had only 60% homology to human GLUT4.

### EXAMPLE 4

### Cloning of GLUT8

Searches of data bases allowed us to obtain an expressed sequence tag (EST) clone which encompassed the sequence which we had obtained by PCR from MCF7 cells. The clone (I.M.A.G.E. Consortium Clone ID 43006), was purchased from Genome Systems, Inc., St. Louis, USA. This clone is 1.2 kb, and includes 500 bp of coding region and a further 700 bp of 3′ UTR sequence. The EST clone was isolated from a neonatal human brain library.

A whole human embryo cDNA λt10 library was a kind gift from B. Kemp. 5×10⁵ plaques were screened. Hybridization was overnight at 42° C. and washes 15 min at 42° C. in 2×SSPE, 0.1% SDS; 30 min at 65° C. in 1×SSPE, 0.1% SDS; and 15 min at room temperature in 0.1×SSPE, 0.1% SDS. cDNAs were cloned into pBluescript SK⁺ (Stratagene). 5′ amplification of cDNA utilized the SMART RACE cDNA Amplification Kit (CLONTECH Laboratories). GLUT8 specific primer (AACATGTACTTCCAGCCAT) was used, followed by a nested PCR reaction with GLUT8 specific forward primer (CGAAGTTTTTCCCCACAC) and reverse primer (TATCTGTCTATCAGGACCCCTCCG). Products were cloned into pGEM-T Easy (Promega), transformed into E. coli strain SURE 2 (Stratagene) and sequenced using the T7 Sequencing Kit (Amersham Pharmacia Biotech), or by Micromon Sequencing Facility (Melbourne, Australia).

Northern blots of RNA extracted from the cultured malignant breast epithelial cell line MCF-7 showed that rat GLUT4 cDNA hybridized under stringent conditions to a transcript of approximately 2.5 kb (data not shown). RT-PCR using GLUT4 specific primers did not amplify a product from MCF-7 RNA. In an effort to identify the transcript that labelled with the GLUT4 probe, we performed degenerate PCR with GLUT4 related primers. The resulting amplification product of approximately 350 bp hybridized to the GLUT4 cDNA by Southern blotting. Sequencing showed 44% identity to human GLUT4 at the amino acid level in the region spanning TM 10 and TM 11. An expressed sequence tag (EST) clone encompassing this sequence was recorded on the public databases and obtained from Genome Systems Inc. (I.M.A.G.E. Consortium [LLNL] Clone ID 43006) (Lennon, 1996). The 1.2 kb EST from a human neonatal brain cDNA library was used to screen a human embryo cDNA library. Although homologous to the GLUT family the positive clones did not contain a complete open reading frame (ORF).

RNA from MCF-7 cells, placenta and human skeletal muscle were used in the first-strand cDNA 5′ RACE reactions. Sequence of four 5′ RACE products overlapped that of partial cDNA clones from the whole human embryonic library. A putative translation start site, which conformed exactly to the Kozak consensus, was identified. A full-length cDNA clone was produced by RT-PCR of RNA from MCF-7 cells and placenta with primers based on putative 5′ and 3′ untranslated region sequence. The cDNA was sequenced completely in both strands. The complete cDNA sequence of GLUT8 is given in SEQ ID NOs: 4 and 30. Nucleotide at position 1129 could not be resolved and is either C (SEQ ID NO: 30) or A (SEQ ID NO:4). The ORF encodes a 617 amino acid polypeptide (SEQ ID NO: 5) with a predicted molecular mass of 66,962 Da.

### EXAMPLE 5

### GLUT8 Genomic DNA

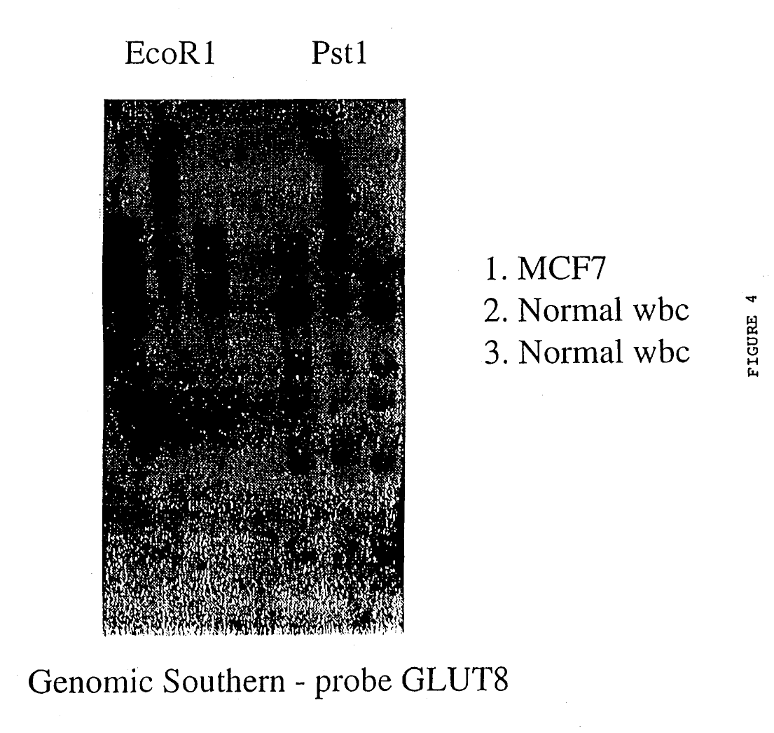

Genomic DNA was extracted from MCF7 cells and whole blood from three human subjects (2 female and 1 male). DNA (20 μg) was digested with restriction enzymes Pst 1, EcoR1, BamH1 or Hind111, separated on agarose gels and transferred to nylon membrane by Southern transfer. Filters were probed with the GLUT8 full-length cDNA probe. Similar restriction patterns were obtained from MCF7 and normal human DNA samples confirming that the GLUT8 gene is present in the normal human genome. The size of the GLUT8 gene is estimated at 15-18 kb. These results are illustrated in FIG. 4.

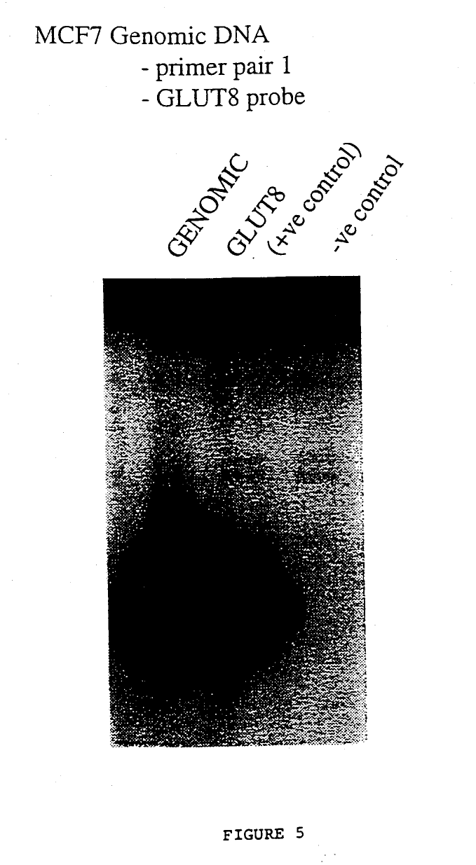

PCR reactions were performed on genomic DNA from MCF7 cells using the GLUT8 specific primer pair 1, as described in Example 7. The PCR product obtained was transferred to nylon membrane, and Southerns blots probed with cDNAs for GLUT1, 2, 3, 4, 5 and 8. Only the GLUT8 probe hybridized to the PCR amplification product obtained with Primer pair 1 as is shown in FIG. 5. These results confirm that the primers used are specific for a gene which is unique from GLUT1, 2, 3, 4 and 5.

### EXAMPLE 6

### Sequence Analysis

The deduced amino acid sequence of the GLUT8 isolated from the embryonic library is set out in SEQ ID NO: 5.

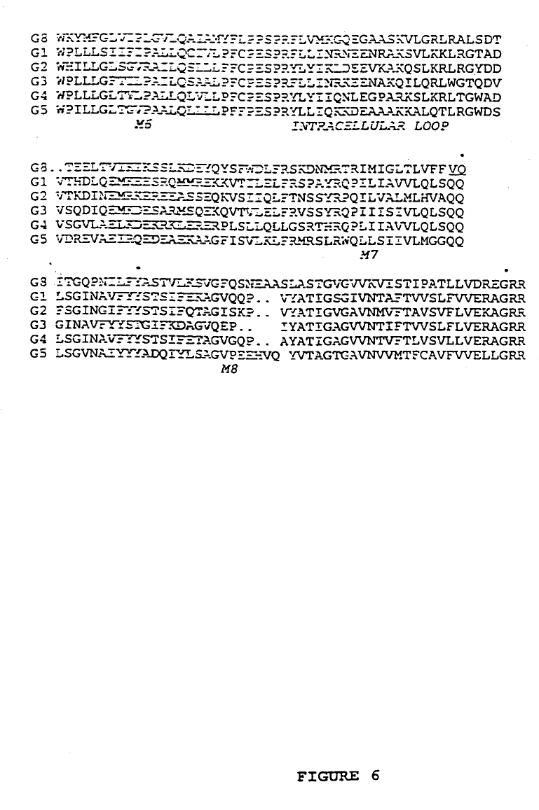

GLUT8 is homologous to the facilitative glucose transporters, as shown in FIGS. 6 and 7, most particularly with 40% amino acid identity to GLUT10. There is homology between proteins from human, bacterium, yeast and plant. Homology to all other members of the glucose transporter family is lower, ranging from 29% amino acid identity (GLUT3, GLUT4 and GLUT8), to 21% (GLUT9). Hydrophobicity plots predict 12 membrane-spanning domains. GLUTs 1 to 5 and GLUT9 possess an N-linked glycosylation site in an extracellular loop between TMs 1 and 2 and glycosylation is thought to be a requirement for glucose transport (Asano, 1991). In contrast, in GLUT6, GLUT8 and GLUT10 the predicted extracellular domain between TMs 1 and 2 is short and these isoforms possess a glycosylation site in an exofacial loop between TMs 9 and 10. Membrane spanning predictions of GLUT8 indicate the absence of an extracellular domain between TMs 1 and 2 and the presence of an exofacial loop between TMs 9 and 10. Like GLUT10, the domain is significantly larger than in the other transporters. GLUT8 possesses several potential sites for N-linked glycosylation in this loop.

The degree of homology, similar predicted protein structures and highly conserved genomic structures suggest that GLUT10 and 12 may represent a separate sub-family of transporters. The cytoplasmic NH2 and COOH termini of GLUT8 are longer than those present in other glucose transporters. The longer cytoplasmic tails along with the predicted large extra-cellular loop denotes GLUT8 as the largest member of the family yet described. GLUT8 contains key signatures found in the larger facilitated sugar transporter family. In helix 7, conserved glutamine and tyrosine residues are present. This region is likely to constitute part of the exofacial, substrate-binding site. The QLS motif found in helix 7, which is conserved in GLUTs 1, 3 and 4 (GLUT1 residues 279-281) is absent from GLUT8. The absence of this motif is important in the kinetics of fructose transport by GLUTs 2 and 5 (Arbuckl, 1996). Sugar release may be controlled by conformational changes in helices 10 and 11 and mutagenesis of GLUT1 has shown that Trp-388 and Trp-412, critical (Garcia, 1992). These residues are also conserved in GLUT8. Motifs important in membrane topology are also conserved. GRR(K) is present between helices 2 and 3 (Baldwin, 1989, Sato, 1999). Between helices 8 and 9 this motif is GSK in GLUT8. The motif EXXXXXXR between helices 4 and 5 and 10 and 11 (Baldwin, 1989) is also conserved.

GLUT8 possesses di-leucine motifs in the NH2 and COOH termini at similar regions to the FQQI and LL targeting motifs in GLUT4 (Piper, 1993, Verhay, 1994). The major site of GLUT4 phosphorylation (serine-488) is adjacent to the di-leucine targeting motif and sequestration of GLUT4 is regulated by phosphorylation at this position (Lawrence, 1990). GLUT8 contains a potential phosphorylation site at serine-11, adjacent to the NH2-terminal di-leucine.

### EXAMPLE 7

### Detection of GLUT8 in Normal and Malignant Tissue

The messenger RNA for GLUT8 has not been detected by Northern blot analysis in any normal adult rat tissue which we have examined so far. Fresh tissues were frozen in liquid nitrogen and total RNA extracted by the method of Chomcynski and Sacchi (1987). The tissues tested were liver, brain, intestine, kidney, testis, heart, skeletal muscle, adipose tissue, and spleen. RNA was separated by denaturing agarose gel electrophoresis, transferred to nylon membrane (Amersham), and hybridized according to the manufacturer's instructions using the GLUT8 cDNA as a probe. In addition GLUT8 was not detected by Northern analysis in RNA from fetal brain, lung, kidney or liver. For these experiments, a fetal human Northern blot (Clonech) was probed with the GLUT8 cDNA.

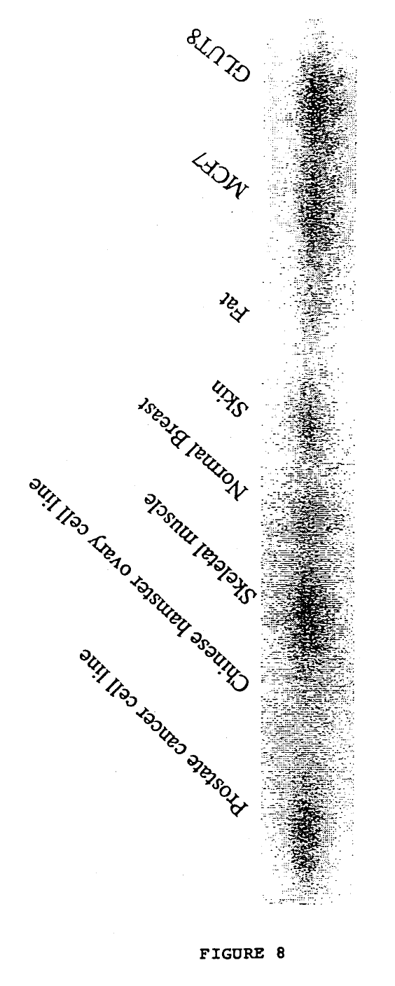

Using primers which are specific for GLUT8, we have detected the presence of GLUT8 by RT-PCR in a human prostate cancer cell line, PC-2, and in human skeletal muscle, skin and to a lesser extent in adipose tissue. Primers and PCR conditions were as described below. The results are shown in FIG. 8. A Chinese hamster ovary cell line gave a negative result.

We have performed semi-quantitative RT-PCR analysis of RNA extracted from 10 human breast cancer samples and from morphologically normal breast tissue taken from the same patients. Samples of tissue were collected at surgery and frozen in liquid nitrogen.

RNA extraction and reverse transcription were performed as described in Example 1.

Primers were designed on the basis of the GLUT8 sequence. PCR was performed with Taq polymerase (Boehringer) using manufacturer's buffer and recommendations. Two primer pairs were used:

| | |
|---|---|
| Primer Pair 1 | |
| Forward primer: | |
| TCCATGGCTGGAAGTACAT | SEQ ID NO: 6 |
| Reverse primer: | |
| TAAGTGTTCTGGCACTATC | SEQ ID NO: 7 |

Primer pair 1 was used in a PCR reaction, the conditions of which were annealing temperature of 50° C. and extension for 1 min for 40 cycles.

| | |
|---|---|
| Primer Pair 2 | |
| Forward primer: | |
| TCAACATCCACATGAACT | SEQ ID NO: 8 |
| Reverse primer: | |
| TGAAAAAGCAGCAACATAAAC | SEQ ID NO: 9 |

Primer pair 2 was used in a PCR reaction, the conditions of which were an annealing temperature of 53° C. and a 30 sec extension for 40 cycles.

These primers are specific for GLUT8, and do not amplify DNA from cDNA constructs of GLUTs 1, 2, 3, 4 or 5. The GLUT1 cDNA probe was obtained from D. E. James (University of Queensland, Australia) and GLUT2, 3 and 5 cDNA probes from G. Bell (University of Chicago, USA).

Amplified PCR products were examined by agarose gel electrophoresis and Southern blots probed with the cDNAs for GLUT4 and GLUT8. The products hybridized only to GLUT8. Normalisation of RNA concentration, RT-PCR reactions and DNA loading were performed by amplification of a non-estrogen-dependent house-keeping gene-36B4 (Labora, 1991). Specific primers for the 36B4 cDNA were synthesised. The PCR reaction was performed with an annealing temperature of 65° C. and extension of 30 secs for 20 cycles.

| | |
|---|---|
| Forward primer: | |
| TGGGCTCCAAGCAGATGC | SEQ ID NO: 10 |
| Reverse primer: | |
| GGCTTCGCTGGCTCCCAC | SEQ ID NO: 11 |

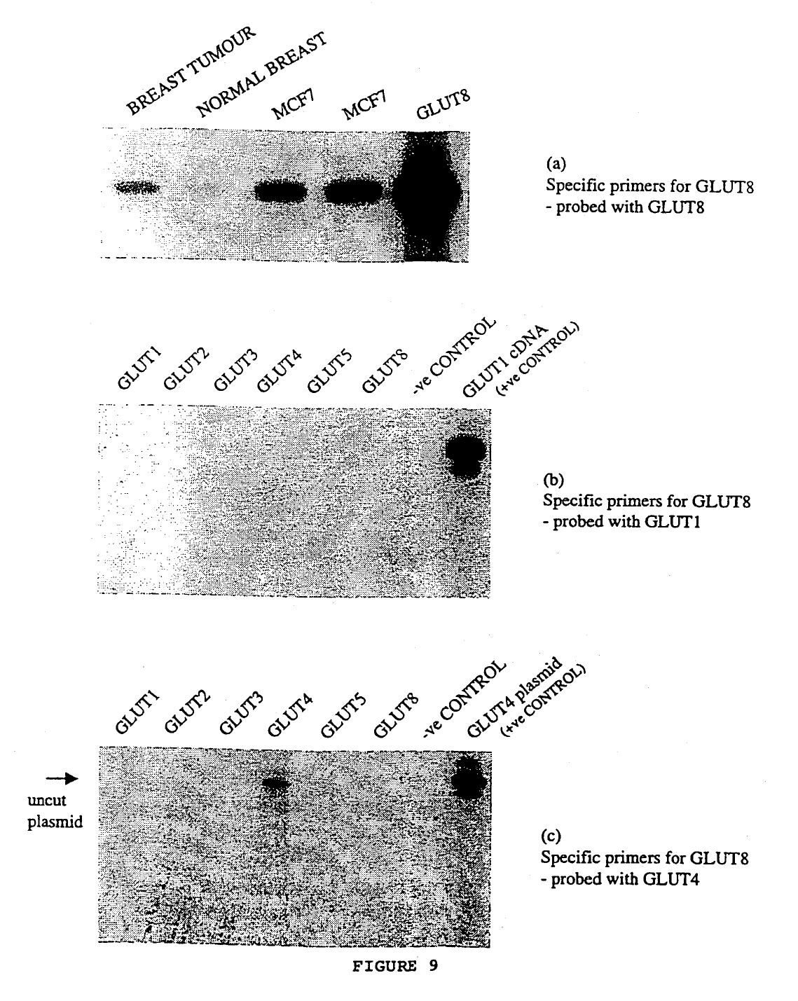

Our preliminary results indicate a higher level of expression of GLUT8 in the tumor tissue compared to normal breast tissue, with GLUT8 being undetectable in some normal samples. This is illustrated in FIG. 9.

### EXAMPLE 8

### Production of Antibody Directed to the C-Terminal Region of GLUT8

We have synthesized a peptide based on the terminal 16 amino acids of the sequence of the C-terminus of GLUT8, as determined from the nucleotide sequence. The C-terminal region has been successfully targeted to produce polyclonal antibodies for GLUTs 1 and 4. A 16 mer peptide

NKLCGRGQSRQLSPET SEQ ID NO: 12

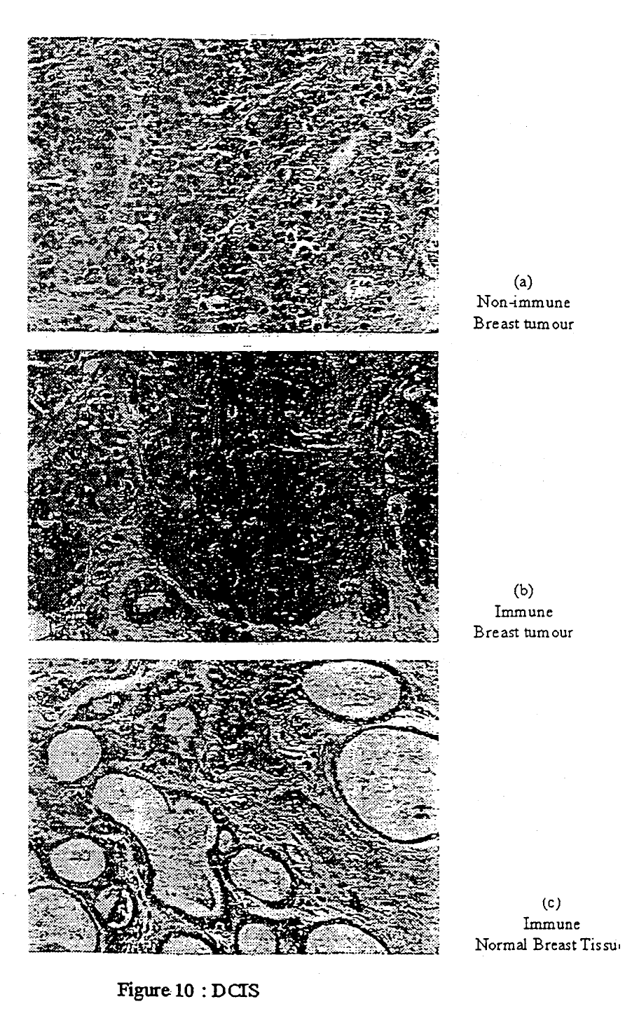

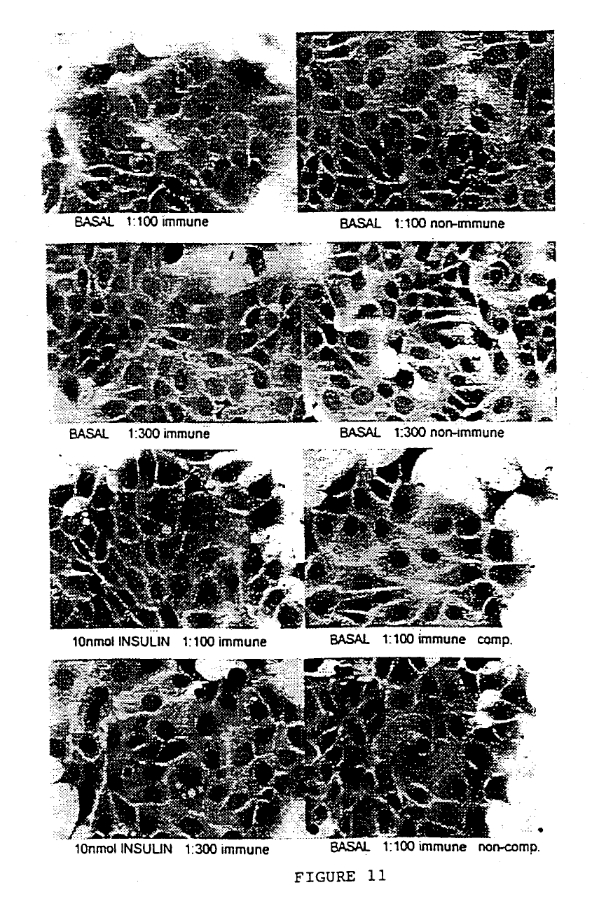

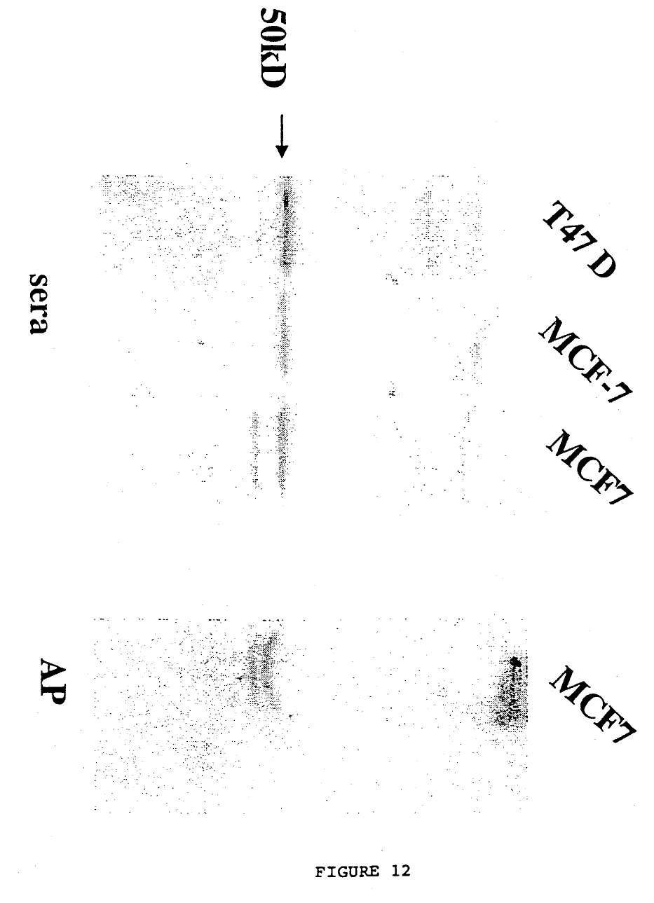

was synthesized, and 2 mg was coupled to 6 mg of N-succinimiyl-3-[2-pyridydithio]propionate (SPDP) activated keyhole limpet hemocyanin (KLM) through the internal cysteine residue according to the manufacturer's instructions (Pierce Chemical Company). Three rabbits were immunized subcutaneously with peptide conjugate (500 μg) emulsified in Freund's colete adjuvant, and boosted at 2 week intervals with peptide conjugate (500 μg) emulsified in Freund's incomplete adjuvant. Final bleeds were taken after the third boost. Anti-serum at a dilution of 1:300 was used for immunocytochemical detection of GLUTS, using the immunoperoxidase reaction in cultured malignant breast cells. Specificity of detection of GLUT8 protein was confirmed by using serial dilutions and comparison to sera from pre-immune bleeds. The results of immunocytochemical detection are illustrated in FIG. 11, which shows that the immune serum at a dilution of 1:100 gave strong staining of MCF7 cells. Serum from a pre-immunization bleed gave a negative result. The immune serum is designated R1396 in subsequent examples. In addition, tumor tissue from a mastectomy sample stained strongly for GLUT8, but normal breast tissue from the same sample was negative, as illustrated in FIG. 10.

### EXAMPLE 9

### Peptide Competition

The specificity of R1396 antiserum for immunocytochemical detection of GLUT8 was further tested in MCF7 cells. Cells were grown, serum starved and fixed as described in Example 17 below. Insulin treatments were for 20 mm at 1 AM. Peroxidase staining was as described in Example 14 except for the omission of H₂O₂ in the detection. Competitive and non-competitive peptides (60 μg/ml in PBS) were incubated on cells for 1 h at room temperature prior to addition of antisera (R1396 or pre-immune bleed) at dilutions of 1/300 or 1/100 containing competitive or non-competitive peptides (final concentration 60 μg/ml) over-night at 4° C. Competitive peptide was that used to immunize and non-competitive peptide EELVPKQPQKRPQELLEC (SEQ ID NO: 29).

These experiments confirm the specificity of R1396 antiserum. GLUT8 staining was observed in the peri-nuclear region of MCF7 cells. Staining pattern was similar but weaker in serial dilutions. No difference in staining pattern was observed following short-term insulin treatment. Staining was competed out by competitive but not non-competitive peptide as shown in FIG. 11.

### EXAMPLE 10

### Western Blot Analysis of the Antibody Raised to the C-terminal Region of GLUT8

A polyclonal antibody specific to the C-terminus of the GLUT8 sequence, was produced and tested in immunohistochemical and immunocytochemical experiments as described above. The antibody, and subsequently the GLUT8 protein, was further characterized by Western blotting experiments and by peptide affinity purification of the antisera.

(a) Affinity Purification

Peptide affinity columns comprising 2 mg of GLUT8 C-terminal peptide per 1 ml column were prepared with SulfoLink Coupling Gel according to the manufacturer's instructions (Pierce Biochemicals, Rockford, Ill.). Purified antibody was eluted with 0.2M glycine, pH 2.0 and dialysed against PBS.

(b) Western Blot Analysis

Crude protein extracts were prepared from MCF7 cultured malignant breast epithelial cells using Trizol reagent (Life Technologies) according to the manufacturer's instructions. Protein was assayed by the BIORAD protein detection method and protein (80 μg), analyzed by SDS PAGE on 12% resolving gel. Proteins were immunoblotted using GLUT8 antisera (1:500) or affinity-purified antibody (50 μgml), and immunolabelled proteins visualized using chemiluminescence detection (Boehringer Mannheim).

Both GLUT8 antisera and affinity-purified antibody immunolabeled a specific protein species with an approximate mobility of 50 kDa, and also labelled a slightly faster-migrating species. These results are shown in FIG. 2. GLUT8 protein has been demonstrated in MCF7 and T47-D cultured malignant breast epithelial cells. The mammalian glucose transporter proteins range from 492 to 524 amino acids (Bell et al., 1990).

### EXAMPLE 11

### Deglycosylation Experiments and Membrane Preparations

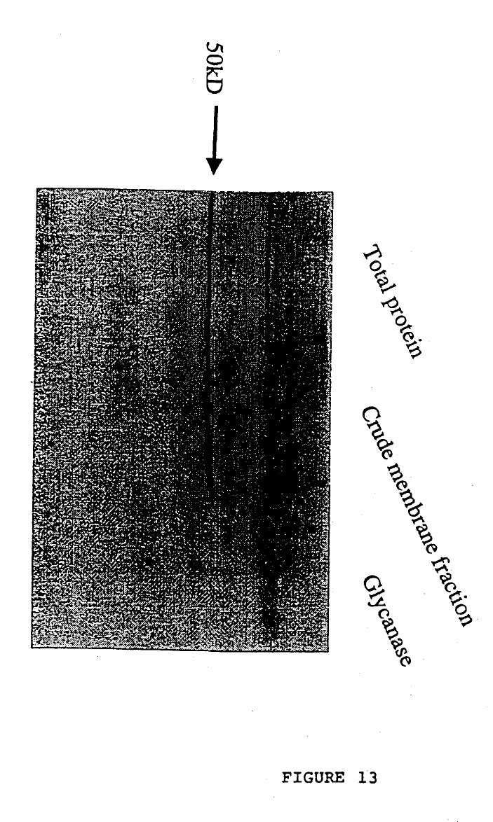

To test the hypothesis that both protein species detected by the GLUT8 antiserum could be different forms of the same protein, protein extracts (80 μg) were treated with endogylcosidase H, 2 mU (Boehringer Mannheim) for 16 h, pH 5.2, 37° C. Western blots of endoglycosidase-treated samples were immunoblotted with GLUT8 antiserum. Only the faster migrating of the two protein species was present in treated samples (FIG. 13), indicating that the GLUT8 protein is glycosylated and that the two proteins detected on Western blots may be different glycosylated forms of the GLUT8 protein. All the members of the facilitative glucose transporter family possess potential sites for N-linked glycosylation. Mutation of Asn-45 of GLUT1 increases the Km for glucose by 2 fold, indicating that glycosylation of the transporter proteins may be necessary for efficient glucose transport (Bell et al. 1993). It is not known at this stage why two potentially altered glycosylated forms of the GLUT8 protein can be detected in MCF7 cells. However, it is noted that during glucose starvation of 3T3-L1 adipocytes, an aglyco form of GLUT1 accumulates (Muekler 1993). The molecular size of GLUT1, which is the predominant transporter in L6 myoblast cells, is greater than that in differentiated myocytes. where GLUT4 is the functional transporter, and this discrepancy is thought to be a result of glycosylation of the GLUT1 protein (Mitsumoto and Klip, 1992).

The glucose transporter proteins are membrane-associated, and can be extracted in membrane fractionation experiments (Bell et al. 1990, Walker et al. 1990). In order to determine whether GLUT8 was present in the membrane fraction of the protein extracts, crude protein extracts from MCF7 cells were precipitated with 1M KCl for 30 mm on ice. Following centrifugation at 14,000 rpm for 30 min, the crude total membrane fraction was solubilzed in detergent (10 mM Tris pH 8.0, 5 mM EDTA, 1 mM phenylmethylsulfonyl fluoride (PMSF), 1% Triton X100).

Membrane fractions were subjected to Western analysis. The GLUT8 polyclonal antibody immunolabels two protein species at approximately 50 kDa in membrane fractions of these cells as shown in FIG. 13.

### EXAMPLE 12

### Cross-Reactivity of GLUT4 Polyclonal Antibody

As discussed above, GLUT8 was unexpectedly identified as a GLUT4-like transporter in malignant breast epithelial cells. In order to clarify whether both GLUT4 and GLUTS were expressed in these cells, a monoclonal antibody directed against GLUT4 (mAb F8; James et al. 1988) was used in Western blot analysis. 1F8 does not detect an immunoreactive protein of 50 kDa in MCF7 protein extracts. Rat gastrocnemius muscle protein extracts were used as a positive control in these experiments, as 1F8 cross-reacts with both human and rat GLUT4 (Fukumoto et al. 1989).

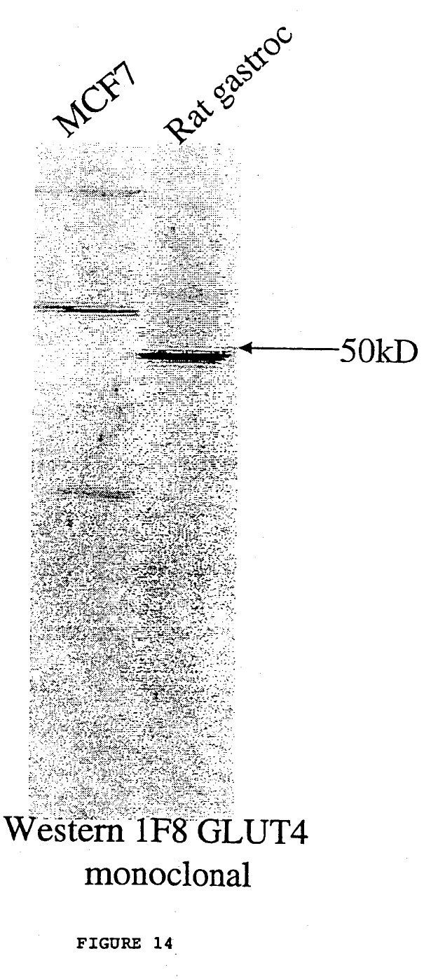

The results of these experiments, illustrated in FIG. 14, suggest that the C-terminal directed GLUT4 polyclonal antibody (R820) cross-reacts with both GLUT4 and GLUT8, but that the GLUT8 polyclonal antibody (R1396) described herein is specific for the GLUT8 protein.

### EXAMPLE 13

### Tissue Distribution of GLUT8 Expression

Because only low levels of GLUTS messenger RNA was detected in all tissues tested, the tissue distribution of expression of GLUT8 was further characterized by Western blot analysis.

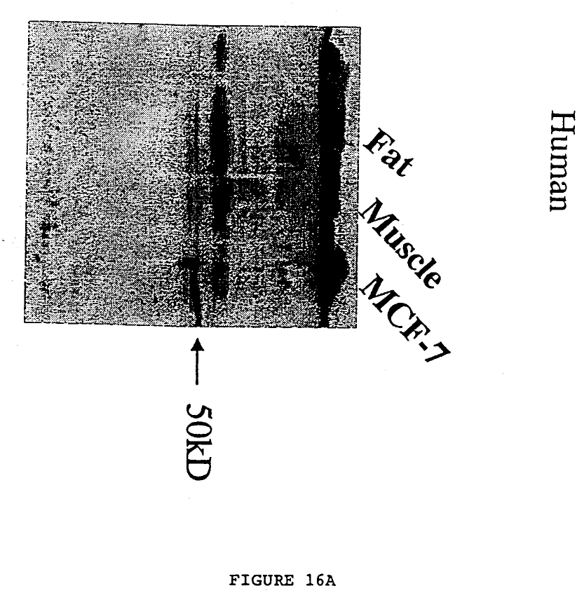

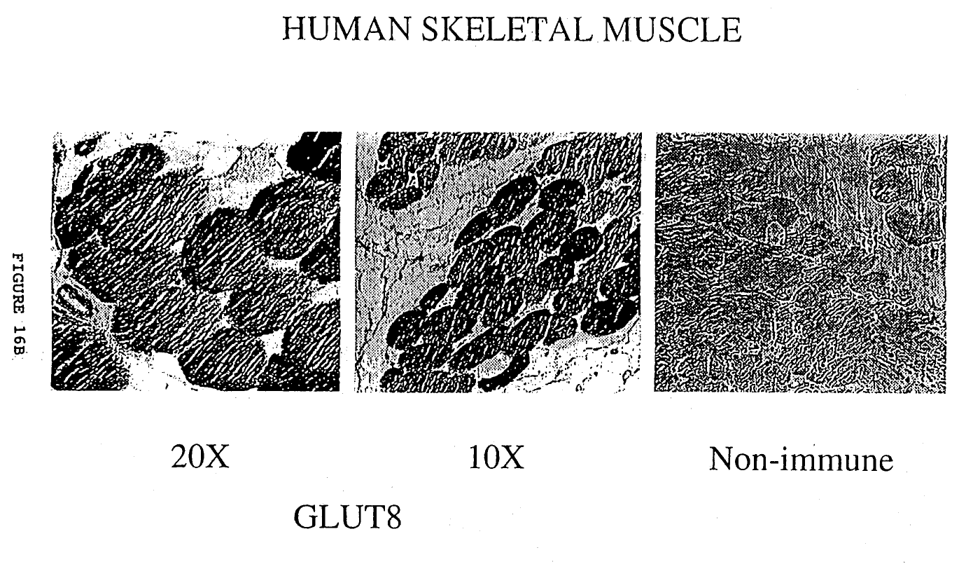

Crude protein extracts were prepared from human tissues using the Trizol extraction method described above. Membrane fractions were prepared as described in Example 11 and immunoblotted with GLUT8 antiserum. We found that GLUT8 protein was present in human adipose tissue and skeletal muscle (FIG. 16a), but not in brain, liver, or kidney. GLUTS immunoreactive protein was also detected in human small intestine protein extracts. It is not known at this stage if the GLUT8 protein in small intestine is present in the mucosa or in smooth muscle remaining after dissection. Our results from immunohistochemical analysis of breast tumor samples suggest that GLUTS is present in smooth muscle cells surrounding blood vessels (Example 14)

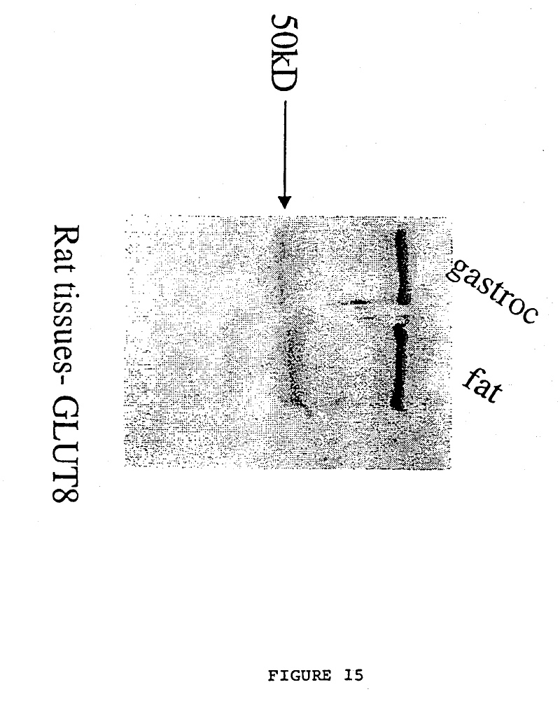

It is of importance for future studies of GLUT8 to determine if the R1396 polyclonal antibody immunolabels GLUT8 protein from other species. Protein was extracted from rat adipose tissue and skeletal muscle (gastrocnemius) by Trizol reagent, and 40 μg protein separated by SDS PAGE. Proteins were immunoblotted with GLUT8 antiserum (1:500). GLUT8 protein was detected in both the rat adipose tissue and skeletal muscle as shown in FIG. 15.

To confirm the presence of GLUT8 protein in skeletal muscle, sections of paraffin-embedded human skeletal muscle (brachioradialis, female) were subjected to immunohistochemical staining with GLUT8 polyclonal antiserum as described in Example 14. Muscle fibre bundles demonstrated strong staining of GLUT8 protein, with no staining by non-immune serum control serum (FIG. 16b).

### EXAMPLE 14

### Use of GLUT8 Antibody for Studying Expression of GLUT8 in Breast Tumors

The histopathology of the breast tumor sample shown in FIG. 10 has been confirmed as ductal cell carcinoma in situ (DCIS). No GLUT4 protein could be detected in this tumor by immunohistochemistry using 1F8 GLUT4 monoclonal antibody. This result is significant in that it indicates that the GLUT8 antibody R1396 is suitable for studying expression of GLUT8 in breast tumor samples. DCIS is a very early stage of breast tumor progression. Detection of GLUTS in this tumor type suggests that GLUT8 may be involved in supply of energy to cells at a very early stage of tumor progression, and ultimately therefore may provide a means of early detection.

In order to investigate this observation further, RT-PCR using specific primer pair 2 was used to compare expression of GLUT8 in 10 breast cancer patients. Sample collection and PCR conditions are as described in Example 7; however the Expand High Fidelity PCR System (Boehringer) was used for amplification of GLUT8. RNA was extracted by Trizol (Life Technologies). In addition, paraffin sections of the tumor samples were stained for immunohistochemical detection of GLUT8 using the standard peroxidase-labelled streptavidin-biotin detection method (Boenisch, 1989). R1396 GLUT8 polyclonal antiserum was incubated on sections overnight, 4° C. at a dilution of 1/300. The peroxidase activity was detected with 3′-3′-diaminobenzidine tetrahydrochioride (Sigma) and H₂O₂ (0.15%). Counterstain was haematoxylin.

Patient demographics are described in Table 1 a and results of RT-PCR and immunohistochemistry summarized in Table 1 b. Comparison of fold increase of GLUT8 expression in tumor tissue over normal breast tissue from the same patient by RT-PCR showed that in 5 of the 10 patients studied, GLUT8 levels were increased in tumor samples. Increases ranged from 3 to 20 fold. Immunohistochemical detection allowed comparison of GLUTS levels in tumor cells and normal ducts at tumor margins. In all but one of the ten patients studied, immunohistochemical and RT-PCR results showed corresponding increases of GLUT8 levels in tumor cells compared to normal ducts. The DCIS component of 6 tumors stained strongly for GLUTS. Smooth muscle cells lining blood vessels were noted to stain positive for GLUT8.

TABLE 1a

| | | Size of | | | | | |
|---|---|---|---|---|---|---|---|
| Patient | Age | Tumor | Nodes ⁺ve | Histology | Grade | E.R. | P.R. |
| 1 | 25 | 12 mm | 0 | APO | 3 | − | − |
| 2 | 42 | 13 mm | 0 | IDC | 2 | + | + |
| 3 | 39 | 29 mm | 2 | IDC | 3 | + | + |
| 4 | 56 | 27 mm | 0 | IDC | 2 | + | + |
| 5 | 85 | 47 mm | 4 | IDC | 3 | − | + |
| 6 | 52 | 10 mm | 0 | LOB | 2 | + | + |
| 7 | 62 | 16 mm | 1 | IDC | 1 | + | + |
| 8 | 77 | 22 mm | 0 | IDC | 2 | + | + |
| 9 | 70 | 38 mm | 15 | IDC | 3 | + | + |
| 10 | 85 | 26 mm | 1 | IDC | 3 | − | − |

Patients demographics
ABBREVIATIONS:
E.R. oestrogen receptor,
P.R.: progesterone receptor
APO: apocrine carcinoma,
IDC: Intraductal carcinoma,
LOB lobular carcinoma

TABLE 1b

| | PCR fold | Invasive | | DCIS | | | |
|---|---|---|---|---|---|---|---|
| Patient | increase | Strength | Area | Strength | Area | Normal | Comments |
| 1 | 1 | 2⁺ | 50% | 2⁺ | 60% | 10% ducts | (myoepithelial cells |
| | | | | | | 10% lobules | staining) |
| 2 | 6 | 1⁺ | 70% | 2⁺ | 70% | 55% ducts | |
| | | | | | | 10% lobules | |
| 3 | 1.4 | 2⁺ | 10% | 1⁺ | 5-10% | -ve | (cysts of the fibrocystic |
| | | | | | | | disease are staining) |
| 4 | 19.7 | 2⁺ | 60% | 2⁺ | 60% | 20% ducts | |
| | | | | | | 20% lobules | |
| 5 | 18.3 | 3⁺ | >90% | 3% | 90% | 40% ducts | |
| | | | | | | 20% lobules | |
| 6 | 1.3 | 1⁺ | 40% | not seen | | 5-10% ducts | (cysts of the fibrocystic |
| | | | | | | 50% lobules | disease are staining) |
| 7 | 3 | 1⁺ | 10-15% | 3⁺ | >90% | 5-10% ducts | |
| | | | | | | 50% lobules | |
| 8 | 1 | 1⁺ | <10% | not seen | | 20% ducts | |
| 9 | 3.6 | 3⁺ | 75% | 3⁺ | 75% | 10% ducts | |
| | | | | | | 50% lobules | |
| 10 | 1 | | 0 | | 0 | 5-10% ducts | |
| | | | | | | 5-10% lobules | |

ABBREVIATIONS:
DCIS ductal cell carcinoma in situ, 1⁺ taint, 2* medium and 3⁺ strong staining

### EXAMPLE 15

### In Vitro Transcription/Translation

The following experiments were performed in order to:

(a) confirm the size of the GLUT8 protein produced from the cloned cDNA; and

(b) confirm that the GLUT8 protein can be glycosylated.

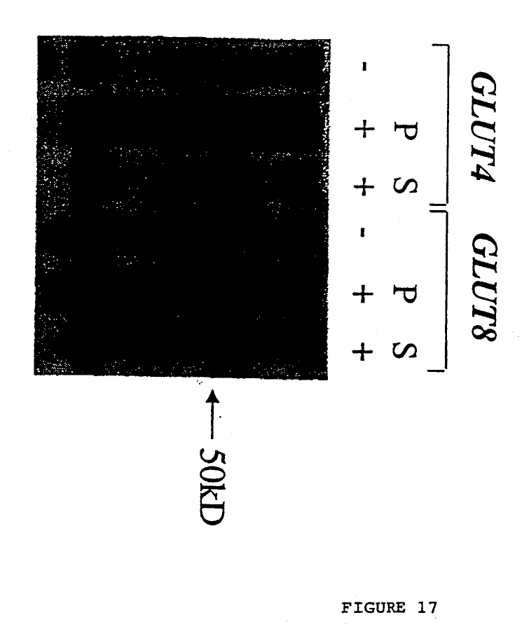

In vitro transcription/translation experiments were performed using a rabbit reticulocyte lysate system (Promega) according to the manufacturer's instructions. In vitro transcription/translation of the GLUT8 cDNA clone yielded a polypeptide of similar mobility (approximately 40 kD) to that produced from rat GLUT4 cDNA. In addition, translation of GLUT4 and GLUT8 mRNA in the reticulocyte lysate system, in the presence of pancreatic microsomal membranes (Promega), reduced the mobility of both protein products, with the larger translation products being present in the pelleted microsome membrane fraction. This is illustrated in FIG. 17. These results suggest that co-translational glycosylation of the translation products had occurred under these conditions, and therefore correlate with our results from the deglycosylation experiments described in Example 11.

### EXAMPLE 16

### Effect of Insulin on Cellular Localization of GLUT8 in MCF7 Cells

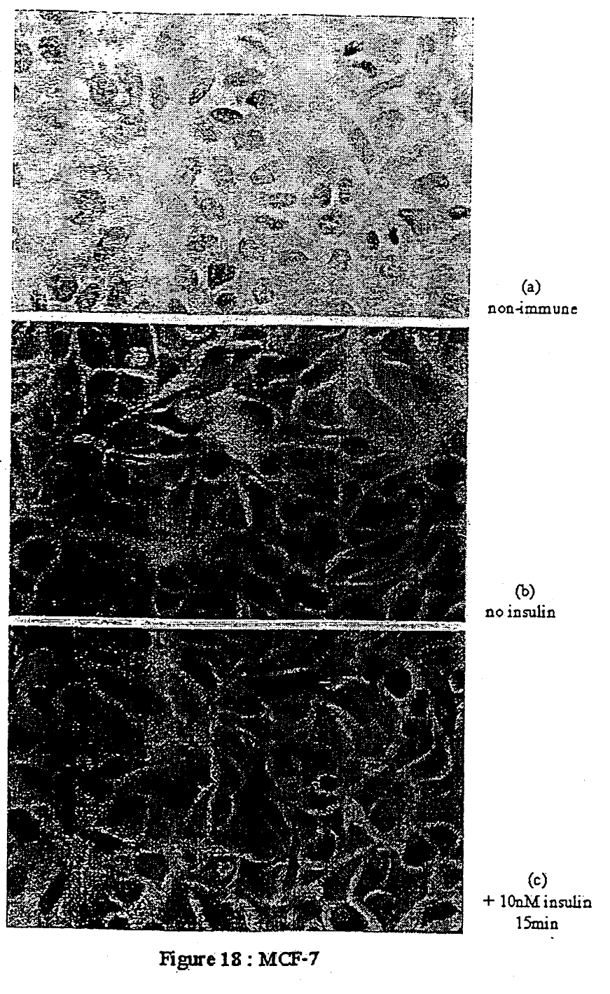

To investigate the effect of insulin on cellular localization of GLUT8 in MCF7 cells, cells were incubated in 10 nM insulin for 15 min. and then subjected to immunocytochemical staining as described in Example 8 and 9. In this case the serum dilution was 1:300. The results are shown in FIG. 18. Non-immune serum again showed no staining, and in the absence of insulin strong staining was observed. After incubation with insulin the staining was diffuse and less intense. It is unclear at this stage whether this indicates movement of GLUT8 into other cell compartments or to the plasma membrane.

### EXAMPLE 17

### Immunofluorescence Studies of GLUT8 Cellular Localisation

As GLUT8 was originally identified on the basis of its homology to GLUT4, and has now been shown to be expressed in insulin-sensitive tissues (skeletal muscle and adipose tissue), we further investigated the potential for translocation of GLUT8 protein in response to insulin treatment. These experiments were performed with cultured malignant breast epithelial cells, but may also be performed with insulin sensitive adipose tissue and muscle cells. Previous results using peroxidase-antiperoxidase staining techniques demonstrated a different staining pattern in NCF7 cells after exposure to insulin. Therefore the more sensitive technique of immunofluorescence was used to determine whether GLUT8 protein is translocated in response to insulin treatment.

Cells were grown in RPMI medium supplemented with 10% Fetal Calf Serum (FCS) and 50 nM insulin. Following fixation with paraformaldehyde (4% in RPMI medium), cells were quenched in glycine (100 mM), permeabilized with 0.1% Triton X-100 amid blocked in horse serum (2%). Cells were incubated overnight at 4° C. with R1396 GLUT8 antiserum (1/300 in 0.2% horse serum), washed with PBS and incubated for 1 h with Texas Red-X goat anti-rabbit IgG, 5μ/ml (Molecular Probes, USA). For short-term insulin treatment, cells were incubated in RPMI supplemented with 2% FCS for 16 h prior to treatment and then in RPMI supplemented with 0.2% BSA for 1 h immediately prior to addition of insulin. For long-term insulin treatment, cells were maintained in RPMI supplemented with 10% FCS and 50 nM insulin. Where the combined effects of serum and insulin were compared to either serum or insulin alone, cells were grown in RPMI with 10% FCS and 50 nM insulin, with media then changed to RPMI with 0.2% BSA, RPMI with 0.2% BSA and 50 nM insulin or RPMI with 10% FCS respectively, and cells incubated for a further 16 h prior to fixation.

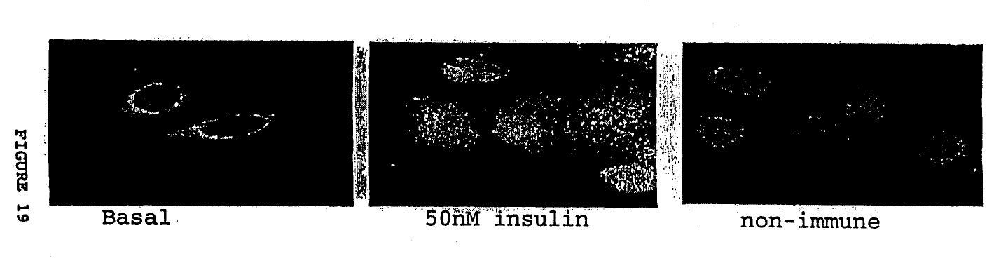

The cellular localization of GLUT8 protein was examined in MCF7 cells by immunofluorescence confocal microscopy. When MCF7 cells were incubated under basal, serum and insulin-starved conditions, staining for GLUT8 was in a tight peri-nuclear pattern with no plasma membrane staining. Growing cells continuously in the presence of insulin (50 nM) and 10% FCS, resulted in a different distribution of GLUT8 protein, with staining throughout the cell and at the plasma membrane. No staining was observed in cells treated with pre-immune serum. These results, shown in FIG. 19, may be indicative of intracellular sequestration of GLUT8 protein in the absence of hormonal stimuli. When MCF7 cells were incubated in either RPMI and 10% FCS or in medium supplemented with insulin alone, the staining pattern observed was similar to that observed under basal, serum and insulin-starved conditions, suggesting that the presence of insulin and an as yet unidentified serum component is required for the redistribution of GLUT8 in these cells.

In insulin-sensitive muscle cells and adipose tissue in the absence of insulin, GLUT4 is sequestered to intracellular compartments. On exposure of these cells to insulin, GLUT4 is rapidly translocated to the plasma membrane, resulting in a rapid and large increase in glucose transport (reviewed by Bell et al. 1993). Insulin has been shown to stimulate cell growth of MCF7 cells via the insulin receptor, which is present at elevated levels in these cells compared to normal breast epithelial cells (Milazzo et al 1992). In our experiments with MCF7 cells we have been unable to demonstrate any acute increase in GLUT8 labeling at the cell surface after exposure to insulin for 15 to 60 minutes at concentrations ranging from 10 nM to 11M. Glucose uptake over this time-frame is elevated 3-fold with insulin treatment, but this relatively small increase could be explained by either increased activity or increased plasma membrane levels of GLUT1, as this GLUT is expressed at high levels in MCF7 cells. However, there is clearly an altered subcellular location of GLUT8 protein when cells are exposed to insulin over longer incubation periods of up to 5 days. Therefore it is possible that this redistribution represents a form of protein trafficking.

Altered cellular localization of GLUT8 in response to insulin and/or other factors may provide a control mechanism for growth of breast cancer cells. In addition we have demonstrated that GLUT8 is present in muscle cells and adipose tissue. These tissues, which express the GLUT4 isoform, are described as classically insulin-responsive. Specific sequences have been identified in the N- and C-terminal regions of GLUT4 which are thought to direct cellular localization and trafficking in response to insulin (Piper et al. 1993). It is thought that reduced insulin-responsiveness in NIDDM may be a result of defective insulin-stimulated translocation of the GLUT4 protein. Much progress has been made in the understanding of the intricate molecular mechanisms which control this process. However, defects in GLUT4 translocation may not be sufficient to cause hyperglycemia. Recent data arising from the study of GLUT4 knock-out mice suggests that a novel insulin responsive glucose transport system may operate in soleus muscle in the absence of GLUT4 expression and under conditions of hyperinsulinemia (Stenbit et al 1996). We have shown that the novel glucose transporter-like protein GLUT8 of this invention is expressed in human skeletal muscle cells and adipose tissue, and therefore could play a role as a second or compensatory insulin responsive transport system. Expression or activity of a second or compensatory insulin stimulated glucose transport system could be altered in insulin resistant NIDDM.

### EXAMPLE 18

### Northern Blot Analysis of MCF7 Cells

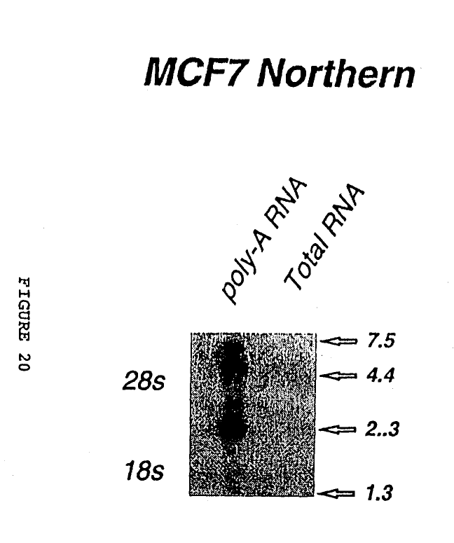

Northern blot analysis of RNA extracted from MCF7 cells detected specific transcripts of approximately 4.4 and 2.5 kb that hybridize to the GLUT8 cDNA, as shown in FIG. 20. The two transcripts that hybridized to the GLUT8 cDNA on Northern blots of MCF7 mRNA may reflect differing lengths of untranslated regions, as has been reported for the human GLUT5 isoform (Kayano et al. 1990). The level of GLUT8 mRNA present in MCF7 cells is low, as transcripts could be detected in polyA but not in total RNA preparations. Whether this result represents a truly low level of mRNA expression in these cells, or whether the message is unstable, or has a high turnover mechanism of regulation, requires further experimentation. However GLUT8 protein is readily detectable in MCF7 cells.

### EXAMPLE 19

### Expression of GLUT8 in Prostate Cancer Cells

RT-PCR was performed on total RNA extracted from cultured prostate cell LNCaP, C4, C4-2, C4-2B using primers to amplify GLUT1, GLUT8 (GLUT8) or the housekeeping gene 36B4. Total protein extracted from prostate cancer cell lines was assessed for GLUT8 protein by Western Blot analysis. Cultured cell monolayers were incubated with antibodies to GLUT1 or GLUT8 and a peripheral Golgi protein, Golgi 58K, for detection by immunofluorescent confocal microscopy. Sections of benign prostatic hyperplasia and human prostate cancer were stained for immunohistochemical detection of GLUT1 and GLUT8.

GLUT1 and GLUT8 mRNA and protein were detected in all cell lines examined. Immunofluorescence staining demonstrated both GLUT1 and GLUT8 on the plasma membrane and the cytoplasm in all cultured prostatic cell lines, with GLUT1 but not GLUT8 appearing to co-localize with the Golgi. Immunohistochemical staining of benign prostatic hyperplasia indicated expression of GLUT1 but not GLUT8. Malignant tissue stained for GLUT8 but was negative for GLUT1.

From these results, it is evident that GLUT8 is expressed in human prostate cancer cells. GLUT8 may play a role in meeting the high energy requirements of these malignant cells.

### REFERENCES

Arbuckle, et al. (1996) Biochemistry 35: 16519-16527

Asano, et al. (1991) J. Biol. Chem. 266: 24632-24636.

Baldwin and PJF Henderson Annu. Rev. Physiol. (1989) 51: 459-471

Bell et al. (1990) Diabetes Care: 13, 198-208.

Bell et al. (1993) J. Biol. Them. :268, 19161-19164

Boenisch, T. (1989) Handbook: Immunochemical Staining Methods, DAKO Corporation

Chomcynski and Sacchi (1987) Anal. Biochem.: 162, 156-159

Fukumoto et al. (1989) J. Biol. Chem. :264, 7776-7779

Garcia et al. (1992) J. Biol. Chem. 267: 7770-7776

Hatt et al. (1992) Strategies: 5, 2-3.

Kahn (1994) Diabetes: 43, 1066-1084.

James et al. (1988) Nature: 333, 183-185

James et al (1989) Nature: 338: 88-87.

Kayano et al. (1990) J. Biol. Chem.: 265, 13276-13282

Keydar et al (1979) Eur. J. Cancer: 15, 659-670.

Labora, J. (1991) Nucl Acids Res.: 19, 3998

Lawrence et al. (1990) J Biol Chem 265: 2324

Lennon et al. Genomics (1996) 33: 151-152.

Mitsumoto and Klip (1992) J. Biol. Chem. :267, 4957-4963

Millazo G (1992) Cancer Res.: 52, 3924-3930.

Muekler (1993) J. Diab. Compl. :7, 130-141

Piper et al. (1993) J. Cell Biol.: 121, 1221-1232.

Sambrook et al. (1989) Molecular Cloning: A Laboratory Manual; Cold Spring Harbor Laboratory Press

Sato and Mueckler (1999) J. Biol. Chem. 274: 24721-24725

Soule et al (1973) J. NatI. Cancer Inst.: 51, 1409-1416.

Stenbit et al (1996) J. Clin. Invest.: 98, 629-634

Thomas et al. (1993) Biochemistry, 30:5139-5145

Verhey and Birnbaum (1994) J Biol Chem, 269, 2353-2356

Walker et al. (1990) J. Biol.: 265, 1516-1523

What is claimed is:

1. An isolated and purified nucleic acid molecule encoding a facilitative glucose transporter protein (GLUT8).

2. The isolated and purified nucleic acid molecule of claim 1, wherein the nucleic acid molecule is SEQ ID NO: 31.

3. The isolated and purified nucleic acid molecule of claim 1, wherein the nucleic acid molecule is SEQ ID NO: 33.

4. The isolated and purified nucleic acid molecule of claim 1, wherein the nucleic acid molecule is SEQ ID NO: 35.

5. The isolated and purified nucleic acid molecule of claim 1, wherein the nucleic acid molecule is SEQ ID NO: 37.

6. The isolated and purified nucleic acid molecule of claim 1, wherein the nucleic acid molecule is SEQ ID NO: 39.

7. The isolated and purified nucleic acid molecule according to claim 1, wherein the nucleic acid molecule is selected from the group consisting of genomic DNA, cDNA, and RNA.

8. The isolated and purified nucleic acid molecule according to claim 1, wherein the nucleic acid is cDNA.

9. The isolated and purified nucleic acid according to claim 1, wherein the nucleic acid has the sequence as set out in SEQ ID NO:4 or SEQ ID NO: 30.

10. An isolated and purified nucleic acid molecule which hybridizes under stringent conditions to the sequence set out in SEQ ID NO: 4 or SEQ ID NO: 30.

11. The isolated and purified nucleic acid molecule according to claim 10, wherein the nucleic acid molecule encodes a protein with 70% amino-acid sequence homology to SEQ ID NO:5.

12. The isolated and purified nucleic acid molecule according to claim 10, wherein the nucleic acid molecule encodes a protein with 80% amino acid sequence homology to SEQ ID NO:5.

13. The isolated and purified nucleic acid molecule according to claim 10, wherein the nucleic acid molecule encodes a protein with 95% amino acid sequence homology to SEQ ID NO:5.

14. A facilitative glucose transporter protein (GLUT8).

15. The facilitative glucose transporter protein according to claim 14, wherein the protein has greater than 70% sequence homology with the amino acid sequence set out in SEQ ID NO:5.

16. The facilitative glucose transporter protein according to claim 14, wherein the protein has greater than 80% sequence homology with the amino acid sequence set out in SEQ ID NO:5.

17. The facilitative glucose transporter protein according to claim 14, wherein the protein has greater than 95% sequence homology with the amino acid sequence set out in SEQ ID NO:5.

18. The facilitative glucose transporter protein according to claim 14, wherein the protein has an amino acid sequence consisting of SEQ ID NO: 32.

19. The facilitative glucose transporter protein according to claim 14, wherein the protein has an amino acid sequence consisting of SEQ ID NO: 34.

20. The facilitative glucose transporter protein according to claim 14, wherein the protein has an amino acid sequence consisting of SEQ ID NO: 36.

21. The facilitative glucose transporter protein according to claim 14, wherein the protein has an amino acid sequence consisting of SEQ ID NO: 38.

22. The facilitative glucose transporter protein according to claim 14, wherein the protein has an amino acid sequence consisting of SEQ ID NO: 40.

23. A method for diagnosing a malignant condition, comprising the steps of:
(a) detecting expression or activity of GLUT8 in a tissue or cell;
(b) correlating the expression or activity of GLUT8 with a malignant condition; and
(c) diagnosing the malignant condition.

24. The method of monitoring of efficacy of treatment of a malignant condition, comprising the step of detecting activity or expression of GLUT8 in a tissue or cell.

25. The method according to claim 23, wherein the method of detection of GLUT8 is selected from the group consisting of immunocytochemistry, hybridization analysis, PCR and RT-PCR.

26. A method of selecting a method of treatment of a malignant condition, comprising the step of measuring the ability of a proposed therapeutic agent to inhibit activity or expression of GLUT8 in a tissue or cell.

27. The method according to claim 26, wherein the inhibition of expression and/or activity of GLUT8 is brought about by either non-utilizable glucose analogues targeted to the malignant tissue or anti-sense nucleic acid sequences directed against the GLUT8 nucleic acid sequence.

28. The method according to claim 24, wherein the tissue or cells are is selected from adipose tissue or skeletal muscle cells.

29. The method according to claim 24, wherein the malignant condition is selected from the group consisting of breast cancer, prostate cancer, epithelial cell cancers such as skin cancers and colon cancers.

30. An antibody directed against GLUT8, or a functional fragment thereof.

31. The antibody according to claim 30, wherein the antibody is either polyclonal or monoclonal.

32. The antibody according to claim 31, wherein the antibody is a polyclonal antibody directed against the C-terminal region of GLUT4.

33. The antibody according to claim 30, wherein the antibody is directed against one or more epitopes present in the sequence set out in SEQ ID NO:12.

34. A method of treating non-insulin dependent diabetes mellitus, comprising the step of upregulating expression of GLUT8 in a tissue or cell.

35. The method according to claim 34, wherein the tissue is skeletal muscle and/or adipose tissue.

36. A method of detecting a mutation in the GLUT8 gene or regulatory sequence of a patient comprising the step of analyzing the gene or regulatory sequence for a nucleic acid change compared to that set out in SEQ ID NO: 3 or 4.

37. The method according to claim 36, wherein the patient is a non-insulin dependent diabetes mellitus patient and the nucleic acid is DNA.

38. The method according to claim 36, wherein the analysis is by single-stranded conformational polymorphism (SSCP).

39. A method of screening putative agents for treatment of cancer, comprising the step of measuring the ability of the agents to inhibit the activity of GLUT8 in vitro or in vivo.

40. The method according to claim 39, wherein the method of screening is positron emission tomography scanning using a hexose labeled with a fluorescent marker.

41. The method according to claim 39, wherein the hexose is either a glucose analogue or hexose specifically transported by GLUT8.

42. A method of screening putative agents for treatment of diabetes and/or insulin-resistance syndrome comprising the step of measuring the ability of the agents to upregulate or enhance the activity of GLUT8 in vitro or in vivo.

43. The method according to claim 42, wherein the diabetes is non-insulin dependent diabetes mellitus.

44. The method according to claim 42, wherein the insulin-resistance syndrome is selected from the group consisting of central obesity, hypertension, dyslypidaemia glucose intolerance.
